(19) United States
(12) Patent Application Publication
(10) Pub. No.: US 2026/0346026 A1
(43) Pub. Date: Nov. 5, 2026

(54) CORONARY SINUS DEPLOYED HEART PACING APPARATUS

(71) Applicant: SHEBA IMPACT LTD., Ramat Gan (IL)

(72) Inventors: Roy BEINART, Ramat Gan (IL); Amit SEGEV, RaAnana (IL)

(21) Appl. No.: 19/123,857

(22) PCT Filed: Nov. 8, 2023
(86) PCT No.: PCT/IL2023/051150

§ 371 Date: Apr. 24, 2025

(30) Foreign Application Priority Data
Nov. 8, 2022 (IL) 298036

(51) Int. Cl. A61N 1/368; A61N 1/05; A61N 1/365; A61N 1/375

(52) U.S. Cl. CPC A61N 1/3684; A61N 1/057; A61N 1/36542; A61N 1/37518; A61N 2001/0585

(57) ABSTRACT

An apparatus for artificial stimulation of the heart of a subject, comprising an elongated body stent-like structure configured to be located in the coronary sinus of the subject, a first electrode assembly mechanically coupled to the body and oriented for electrifying the left atrium of the subject when the body is located in the coronary sinus, a second electrode assembly mechanically coupled to the body sufficiently distant from the first electrode assembly and oriented for electrifying the left ventricle of the subject when the body is located in the coronary sinus and the first electrode assembly is oriented for electrifying the left atrium, and a controller assembly electrically coupled to the first electrode assembly and to the second electrode assembly, the controller assembly is configured to drive a pacing electrical signal to the first electrode assembly and/or to the second electrode assembly.

### RELATED APPLICATIONS

This application claims the benefit of priority of Israel Patent Application No. 298036 filed on Nov. 8, 2022, the contents of which are incorporated herein by reference in their entirety.

### FIELD AND BACKGROUND OF THE INVENTION

The present invention, in some embodiments thereof, relates to a heart pacing apparatus and, more particularly, but not exclusively, to a heart pacing apparatus deployed in the coronary sinus of a subject for electrifying the left atrium and/or the left ventricle of the subject.

The heart is a muscular pump, which uses electrical signals to control its synchronization. Many cardiac disorders are caused by or manifest as incorrect timing of contraction of heart chambers.

A common solution is a using a pacemaker, which provides an electrical signal to cause activation of the hearts or parts thereof at a desired timing.

Wang ZHAOJUN et. al proposes one such solution is by in Chinese Patent Publication No. CN111939476(A) titled “USING METHOD OF LEADLESS DOUBLE-CAVITY PACEMAKER SYSTEM PLACED IN CORONARY SINUS” which discloses a leadless double-cavity pacemaker system placed in a coronary sinus for realizing leadless atrial and ventricular double-cavity pacing and sensing without damaging myocardium.

### SUMMARY OF THE INVENTION

Some embodiments of the invention provide, methods, systems and/or software program products for stimulating a heart of a subject using an apparatus which is located in the coronary sinus of the subject and capable of electrifying the left atrium and/or the left ventricle of the subject. This may be provided by the features of the independent claims. Further implementation forms may be apparent from the dependent claims, the description and the figures.

According to a first aspect of the present invention there is provided an apparatus for artificial stimulation of the heart of a subject, comprising an elongated body having a cylindrical structure with flow-lumen therein configured to be located in the coronary sinus of the subject, a first electrode assembly mechanically coupled to the body and oriented for electrifying the left atrium of the subject when the body is located in the coronary sinus, a second electrode assembly mechanically coupled to the body sufficiently distant from the first electrode assembly and oriented for electrifying the left ventricle of the subject when the body is located in the coronary sinus and the first electrode assembly is oriented for electrifying the left atrium, and a controller assembly electrically coupled to the first electrode assembly and to the second electrode assembly, the controller assembly is configured to drive a pacing electrical signal to the first electrode assembly and/or to the second electrode assembly. Wherein the first electrode assembly and/or the second electrode assembly penetrate the left atrium and/or the left ventricle respectively.

According to a second aspect of the present invention there is provided a method of artificially stimulating the heart of a subject, comprising providing an apparatus located in the coronary sinus of the subject, the apparatus comprises a controller assembly, a first electrode assembly for electrifying the left atrium of the subject and a second electrode assembly for electrifying the left ventricle of the subject, the controller assembly is configured to drive a pacing electrical signal to the first electrode assembly and/or to the second electrode assembly.

According to a third aspect of the present invention there is provided a kit for artificial stimulation of the heart of a subject, comprising an apparatus according to the first aspect, and an intravenous delivery system comprising a guiding element shaped to mechanically interlock with the body and operable to rotate the body for placing the body in a determined angular positon around a longitudinal axis of the body.

According to a fourth aspect of the present invention there is provided a method of positioning a heart stimulation apparatus in a coronary sinus of a subject, comprising mapping a location of the left atrium and the left ventricle of the subject, operating an intravenous delivery system to deliver a heart stimulation apparatus to the coronary sinus of the subject, mapping a stimulation effect of muscle tissue of the left atrium and/or the left ventricle based on analysis of a response to stimulation electrical signals injected at a plurality of locations of the left atrium and/or the left ventricle using one or more mapping elements coupled to the intravenous delivery system, selecting an angular position of a guiding element of the intravenous delivery system interlocked with the heart stimulation apparatus according to the mapping, operating the intravenous delivery system to rotate the guiding element according to the selected angular position to position the heart stimulation apparatus in a determined angular position with respect to the left atrium and the left ventricle, and operating the intravenous delivery system to place the body in the coronary sinus in the determined angular position such that a first electrode and/or a second electrode of the heart stimulation apparatus penetrate the left atrium and/or the left ventricle respectively.

According to a fifth aspect of the present invention there is provided a device for intravenously retrieving a battery of an apparatus for artificial stimulation of the heart of a subject, comprising a battery retrieval element operable to release one or more batteries of a heart stimulation apparatus by unlocking a snap-fit element attaching the one or more batteries to a capsule of the apparatus, and retrieve the released one or more batteries.

According to a sixth aspect of the present invention there is provided a method of retrieving a battery of an apparatus for artificial stimulation of the heart of a subject, comprising operating an intravenous system to position a battery retrieval device with respect to a heart stimulation apparatus located in the coronary sinus of a subject, operating a battery retrieval element of the battery retrieval device to release one or more batteries of the apparatus by unlocking a snap-fit element attaching one or more batteries of the apparatus to a capsule of the apparatus, and operating a battery retrieval element to retrieve the released one or more batteries.

In an optional implementation form of the first, second, third and/or fourth aspects, the controller assembly is configured to receive cardiac pacing signal sensed via the first electrode assembly and/or the second electrode assembly, and drive the pacing electrical signal to the first electrode assembly and/or to the second electrode assembly according to the sensed cardiac pacing data.

In a further implementation form of the first, second, third and/or fourth aspects, the first electrode assembly comprises a plurality of atrial struts extending from the body for penetrating through a wall of the coronary sinus into muscle tissue of the left atrium and the second electrode assembly comprises a plurality of ventricular struts extending from the body for penetrating through a wall of the coronary sinus into muscle tissue of the left ventricle, each of the plurality of struts comprises one or more electrical leads for delivering the pacing electrical signal.

In a further implementation form of the first, second, third and/or fourth aspects, the plurality of atrial struts extend from the body in a first radial direction with respect to a longitudinal axis of the body and the plurality of ventricular struts extend from the body in a second radial direction with respect to a longitudinal axis of the body, the first radial direction is offset from the second radial direction to orient the plurality of atrial struts to extend towards the left atrium and orient the plurality of ventricular struts towards the left ventricle when the body is located in the coronary sinus.

In a further implementation form of the first, second, third and/or fourth aspects, the plurality of atrial struts extend from the body in a plurality of first radial directions covering a predefined first circumferential sector and the plurality of ventricular struts extend from the body in a plurality of second radial directions covering a predefined second circumferential sector, the first circumferential sector is offset from the second circumferential sector to orient the plurality of atrial struts to extend towards the left atrium and orient the plurality of ventricular struts to extend towards the left ventricle when the body is located in the coronary sinus.

In a further implementation form of the first, second, third and/or fourth aspects, the first circumferential sector and/or the second circumferential sector cover a range of 120-360 degrees.

In a further implementation form of the first, second, third and/or fourth aspects, one or more of the electrical leads of one or more of the plurality of atrial struts and/or ventricular struts is disposed externally on the one or more struts.

In a further implementation form of the first, second, third and/or fourth aspects, one or more of the electrical leads of one or more of the plurality of atrial struts and/or ventricular struts is extending from an interior bore of the one or more struts.

In a further implementation form of the first, second, third and/or fourth aspects, the plurality of atrial struts and ventricular struts are oriented to extend from the body in a common axial direction with respect to a longitudinal axis of the body.

In a further implementation form of the first, second, third and/or fourth aspects, each of the plurality of atrial struts and ventricular struts is disposed to extend from the body in one of a plurality of axial directions with respect to a longitudinal axis of the body.

In a further implementation form of the first, second, third and/or fourth aspects, at least some of the plurality of struts are shaped for anchoring the body in place when located in the coronary sinus.

In an optional implementation form of the first, second, third and/or fourth aspects, the apparatus comprises one or more anchor elements for anchoring the body in place when located in the coronary sinus.

In a further implementation form of the first, second, third and/or fourth aspects, the body has is a stent-like structure.

In a further implementation form of the first, second, third and/or fourth aspects, the body is constructed of a plurality of separate segments mechanically coupled to each other via one or more coupling elements.

In a further implementation form of the controller assembly is encapsulated in a capsule mechanically coupled to the body, the capsule comprises one or more batteries electrically connected to the controller assembly for powering the controller assembly.

In a further implementation form of the first, second, third and/or fourth aspects, one or more of the batteries are detachably attached to the capsule via a releasable snap-fit element.

In a further implementation form of the first, second, third and/or fourth aspects, one or more of the batteries are rechargeable.

In an optional implementation form of the first, second, third and/or fourth aspects, the controller assembly further comprises one or more motion sensors operable to capture motion data, the controller assembly is configured to adjust the pacing electrical signal according to the captured motion data.

In a further implementation form of the first, second, third and/or fourth aspects, the guiding element comprises mechanical provisions shaped to receive and accommodate mating mechanical provisions disposed on the body and interlock with the body in a rotational axis around the longitudinal axis of the body to induce rotation of the body when rotating the guiding element.

In a further implementation form of the first, second, third and/or fourth aspects, the mechanical provisions of the guiding element comprise one or more depressions and the mating mechanical provisions of the body comprise one or more protrusions shaped to fit into the one or more depressions and/or vice versa.

In an optional implementation form of the first, second, third and/or fourth aspects, the guiding element comprises a limiting element disposed at a distal end of the guiding element for limiting a movement of the body along its longitudinal axis.

In an optional implementation form of the first, second, third and/or fourth aspects, the intravenous delivery system comprises one or more mapping elements comprising a plurality of mapping electrodes configured to drive stimulating electrical signals to the left atrium and/or to the left ventricle, a muscular activity of the left atrium and/or to the left ventricle in response to the stimulating electrical signals is analyzed to map stimulation effect of the stimulating electrical signals.

In an optional implementation form of the first, second, third and/or fourth aspects, the intravenous delivery system comprises an over tube shaped to accommodate the body at least while moved intravenously to the coronary sinus.

In a further implementation form of the fifth and/or sixth aspects, the battery retrieval element is shaped to attach to one or more mating elements disposed on the one or more batteries which is shaped for locking to the capsule via the snap-fit element.

In an optional implementation form of the fifth and/or sixth aspects, the battery retrieval element is operated to attach one or more new batteries to the capsule via the snap-fit element.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks automatically. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of methods and/or systems as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars are shown by way of example and for purposes of illustrative discussion of embodiments of the invention. In this regard, the description taken with the drawings makes apparent to those skilled in the art how embodiments of the invention may be practiced.

In the drawings:

FIG. 1A and FIG. 1B are schematic illustrations of an exemplary heart stimulation apparatus designed and configured for deployment in the coronary sinus of a subject for artificially pacing the left atrium and/or the left ventricle of the subject, according to some embodiments of the present invention;

FIG. 2A and FIG. 2B are schematic illustrations of exemplary embodiments of a heart stimulation apparatus designed and configured for deployment in the coronary sinus of a subject for artificially pacing the left atrium and/or the left ventricle of the subject, according to some embodiments of the present invention;

FIG. 3A and FIG. 3B are schematic illustrations of a front view of an exemplary heart stimulation apparatus designed and configured for deployment in the coronary sinus of a subject for artificially pacing the left atrium and/or the left ventricle of the subject, according to some embodiments of the present invention;

FIG. 4A and FIG. 4B are schematic illustrations of exemplary deployments of struts extending from a heart stimulation apparatus for electrifying the left atrium and/or the left ventricle of a subject, according to some embodiments of the present invention;

FIG. 5A is a flowchart of an exemplary process for placing a heart stimulation apparatus in the coronary sinus of a subject, according to some embodiments of the present invention;

Reference is now made to FIG. 5B and FIG. 5C, which are schematic illustrations of an exemplary intravenous delivery system adapted for delivering and deploying a heart stimulation apparatus in the coronary sinus of a subject, according to some embodiments of the present invention;

FIG. 6 presents schematic illustrations an exemplary guiding element configured and operable for placing an exemplary heart stimulation apparatus in the coronary sinus of a subject, according to some embodiments of the present invention;

FIG. 7A and FIG. 7B are schematic illustrations of exemplary mapping elements of an intravenous delivery system configured and operable for placing an exemplary heart stimulation apparatus in the coronary sinus of a subject, according to some embodiments of the present invention;

FIG. 8 is a flowchart of an exemplary process for artificially stimulating the left atrium and/or the left ventricle of a subject using a heart stimulation apparatus deployed in the coronary sinus of the subject, according to some embodiments of the present invention;

FIG. 9A, FIG. 9B and FIG. 9C are schematic illustrations of an exemplary battery retrieval device configured and operable for retrieving one or more batteries of an exemplary heart stimulation apparatus deployed in the coronary sinus of a subject, according to some embodiments of the present invention; and

FIG. 10 is a flowchart of an exemplary process for replacing one or more batteries of an exemplary heart stimulation apparatus deployed in the coronary sinus of a subject, according to some embodiments of the present invention.

### DETAILED DESCRIPTION OF EMBODIMENTS OF THE INVENTION

The present invention, in some embodiments thereof, relates to a heart pacing apparatus and, more particularly, but not exclusively, to a heart pacing apparatus deployed in the coronary sinus of a subject for electrifying the left atrium and/or the left ventricle of the subject.

According to some embodiments of the present invention there is provided a heart artificial stimulation apparatus (artificial cardiac pacemaker) for artificially stimulating of the heart of a subject, for example, a human, a mammal, and/or the like.

In particular, the artificial stimulation apparatus is optionally configured, shaped and/or adapted to be located (deployed) in the coronary sinus of the subject for artificially pacing the left atrium and/or the left ventricle of the subject.

The artificial stimulation apparatus may comprise an elongated body which may extend in the coronary sinus between the left atrium and the left ventricle of the subject. Specifically, the body of the apparatus may extend between muscle tissue areas (myocardium) of the left atrium and the left ventricle which are more effective for pacing that locations which directly face each other.

In some embodiments of the invention, the artificial stimulation apparatus comprises two electrode assemblies. Each assembly includes one or more electrodes. In some embodiments of the invention, each assembly acts as a bi-or multi-polar electrode. Optionally or additionally, a common return electrode may be provided on the apparatus (e.g., on a circuitry housing thereof. In some embodiments of the invention, the electrodes are arranged to exit the coronary sinus and extend towards or into the muscle of the heart.

Moreover, the electrode assemblies may comprise penetrating electrodes adapted to penetrate the muscle tissue of the left atrium and the left ventricle to effectively sense signals and/or electrify the left atrium and the left ventricle. For example, each assembly may include a plurality of micro-needle like struts (studs) extending outwards from the body of the apparatus. The first electrode assembly may be electrified with pacing electrical signal for artificially pacing (electrifying) the left atrium while the second electrode assembly may be electrified with pacing electrical signal for artificially pacing the left ventricle. In some embodiments, one or more electrodes in one or both assemblies (optionally same ones as used for electrifying the heart) are used for sensing.

Since the more effective pacing areas in the myocardium in the left atrium and in the left ventricle, may be relatively distant, the first and second electrode assemblies may be mechanically coupled to the body sufficiently distant from each other.

Moreover, since the effective pacing areas of the left atrium and in the left ventricle may circumferentially (angularly) offset from each other with respect to a longitudinal axis of the coronary sinus, the first electrode assembly may be oriented to extend the atrial struts in a first radially directed angle and the second electrode assembly may be oriented to extend the ventricular struts in a second radially directed angle. In some embodiments of the invention, the struts of one or both assemblies extend for a range of different angles. Optionally or additionally, the struts of one or both assemblies extend over a range of axial positions.

As such, when the body of the apparatus is positioned and oriented in the coronary sinus in the determined position (and orientation), the atrial struts may penetrate through the wall of the coronary sinus into the left atrium, specifically into muscle tissue of the left atrium at a desired effective pacing area while the ventricular struts may penetrate through the wall of the coronary sinus into the left ventricle, specifically into muscle tissue of the left ventricle at a desired effective pacing area.

The artificial stimulation apparatus may comprise a controller assembly typically encapsulated in a housing, for example, a capsule which is electrically coupled to the first and second electrode assemblies. The controller assembly may comprise a signal generator for driving pacing electrical signals to the first electrode assembly for pacing the left atrium and/or to the second electrode assembly for pacing the left ventricle.

Optionally, the controller assembly may be configured to drive, adjust, and/or inhibit the pacing electrical signal driven to the first and/or second electrode assemblies according to cardiac signals sensed in the left atrium and/or in the left ventricle via the struts of the first and/or second electrode assemblies.

The apparatus may comprise one or more batteries mechanically and electrically connected to the capsule of the apparatus for powering the control assembly.

Optionally, one or more of the batteries may be detachably attached to the capsule. Optionally, attachment is via one or more releasable snap-fit elements such that they may be replaced while in the apparatus is located in the body of the subject, in particular in the coronary sinus. Optionally, one or more of the batteries may be rechargeable while in the apparatus is located in the body of the subject, in particular in the coronary sinus.

The coronary sinus deployed heart stimulation apparatus may present major advantages and benefits compared to existing heart stimulating and pacemaker devices and/or may overcome limitations inherent to such existing devices.

Traditional pacing devices typically comprise a pulse generator implanted under the skin of the subject, most commonly in the left anterior chest wall, which drives pacing electrical signals to one or more of the heart's chambers via pacing leads going into the subject's heart through the superior vena cava. Such pacemakers, for example, single chamber pacemakers, dual chamber pacemakers, and biventricular pacemakers, also known as Cardiac Resynchronization Therapy (CRT), may have major limitations.

First, the pacing leads placed in the heart chambers are susceptible to displacement which may significantly reduce their pacing efficiency. In addition, coming through the superior vena cava, the pacing lead going into the right ventricle (typically to pace the left ventricle via the right ventricular septum) passes through the tricuspid valve controlling blood flow from the right atrium to the right ventricle. This right ventricle pacing lead may degrade operation of the tricuspid valve since it may not fully close to prevent blood from going back into the right atrium. Such leads are also at risk of adhesion and/or bacterial growth. On top of this, reaching a pacing location in the left ventricle is not easy. A potential advantage of the coronary sinus deployed heart stimulation apparatus, is pacing of the left ventricle at desired locations thereof while not requiring leads.

Moreover, the pacing leads of the traditional pacemakers are placed inside the chambers and the pacing electrical signals they drive for stimulating the muscle tissue of the respective chamber are often not well aligned and synchronized with the origin of natural cardiac pacing signals which originate a base of the muscle tissue (myocardium) outside the chamber. As such the pacing signals driven by the traditional pacemakers may be less effective and may even damage and/or degrade the chamber's muscle. A potential advantage of the coronary sinus deployed heart stimulation apparatus may be its ability to apply physiological pacing to the left ventricle which is highly aligned with the natural cardiac pacing signals stimulating the chamber's muscle. This is because the ventricular struts of the coronary sinus deployed heart stimulation apparatus may penetrate the muscle tissue in close proximity to the natural conduction system conveying the natural cardiac pacing signals stimulating the chamber's muscle. The pacing electrical signals generated by the coronary sinus deployed heart stimulation apparatus may therefore significantly improve the pacing performance and reduce or potentially avoid damage to the chambers' muscles.

Furthermore, the coronary sinus deployed heart stimulation apparatus may stimulate the subject's heat with significantly lower amplitude pacing signals compared to the legacy pacing devices since the atrial and ventricular struts may penetrate the muscle tissue of the left atrium and/or the left ventricle in close proximity to the natural cardiac pacing signals center which stimulating the chamber's muscle. For example, due to the fact that electrodes of at least some of the existing pacing devices are placed on the inner wall of the chambers (e.g., atrium and/or ventricle), the pacing signals they drive to stimulate the chamber's muscle are significantly high energy signals with amplitudes reaching 3 Volt (V) and even 5V in some cases. In contrast, low energy pacing signals having an amplitude which may be as low as 0.5V driven by the coronary sinus deployed heart stimulation apparatus may be at least as effective as the legacy pacemakers and typically more effective.

Reducing the signals' energy may significantly reduce the power consumption of the coronary sinus deployed heart stimulation apparatus which may therefore require less maintenance (e.g. battery charging or replacement). In addict, reducing the signal's energy may reduce potential damage to the chamber's muscle tissue which may significantly degrade when injected with high energy pulses as may be done by the existing pacemakers.

Cardiac stimulation devices can cause pain and/or other discomfort. This may be caused by the physical location of the device and/or due to electrification of other tissue. A potential advantage of the coronary sinus deployed heart stimulation apparatus is avoidance of such physical location and/or less electrification of tissue which does not need to be electrified.

More recent leadless pacing technology may be based on a pacing capsule deployed in the right ventricle for stimulating it. Such capsule pacemakers which are deployed in the ventricle may be unable to effectively synchronize pacing signals between the atrium and the ventricle, for example, the between the right atrium and the right ventricle.

A potential benefit of a coronary sinus deployed heart stimulation apparatus according to some embodiments of the invention is synchronizing between the activation of the left atrium and left ventricle, for example by synchronized stimulation or by stimulating one based on measurements from the other. In some embodiments of the invention, a more global sensing of electrical activity in the heart is provided by sensing between an electrode in the first assembly and an electrode in the second assembly (or between one assembly and the circuitry capsule or other electrode). The distance between such electrodes can be large enough to allow sensing electrical activity of the heart as a whole.

A particular feature of some embodiments of the invention is that the apparatus is implanted as a single integral unit. One potential advantage is easy of implantation. Another potential advantage is ease of maintenance—as there need be only on circuitry capsule and/or power supply and/or telemetry circuit. Another potential advantage is easy and/or reliability of synchronization between different parts of the apparatus.

Another potential advantage is lower power requirements as no power need be wasted on wireless communication between sub-components. Another potential advantage is reliability as there are fewer component for failure as compared to a multi-part system. In some embodiments however, the apparatus as described may be implanted to work with additional implanted devices, for example, another pacemaker, an Implantable Cardioverter-Defibrillator (ICD), and/or the like and receive pacing signals, and/or coordinate pacing signals using a wireless transmitter.

According to some embodiments of the present invention there is provided an optional intravenous delivery system configured to position the heart stimulation apparatus in the coronary sinus. In some embodiments of the invention, the delivery system includes a guiding means configured to control an axial and/or rotational position of the apparatus. In one example, the guiding means is used for rotating the apparatus or is rotated manually and then the apparatus is delivered, specifically to rotate the body of the apparatus to orient it in a determined angular position around the longitudinal axis of the body, optionally so that electrodes of the apparatus are aligned with locations to be sensed and/or paced, before anchoring the apparatus in the determined location in the coronary sinus.

Using the guiding means to rotate the body of the heart stimulation apparatus and accurately orient it in the coronary sinus may enable accurate orientation of the atrial struts and the ventricular struts to extend towards the left atrium and the left ventricle respectively. Then, when anchoring the heart stimulation apparatus in the coronary sinus, the atrial struts and the ventricular struts may penetrate into the muscle tissue of the left atrium and the left ventricle respectively at the areas which are estimated to be effective, safe and/or satisfactory for artificial stimulation. Optionally or additionally, electrode penetration into other tissue, or away from the heart is avoided.

In some embodiments of the invention, the guiding system includes a geometry which interferes with eth apparatus to prevent relative rotation thereof, for example a groove in the guiding means and a protrusion I the apparatus. Optionally or additionally, the guiding means includes a stop, for example a section greater in diameter than a part of the apparatus, so that axial advance of the apparatus is stopped when the apparatus reaches the stop.

In some embodiments of the invention, a mapping system is provided, which mapping system is used to electrically map tissue adjacent the coronary sinus, in order to identify locations where electrode placement is desirable and/or undesirable.

A potential benefit of having a guiding system together with a mapping system is that a user can map the cardiac tissue near the coronary sinus to identify a location and use the same mechanical system used for mapping, in order to implant and position the coronary sinus pacing apparatus. This can provide repeatability, effectiveness and/or ease of use.

In some embodiments of the invention, the guiding system is used to orient and/or axially position the mapping system. Optionally or additionally, the guiding system is advanced to be aligned with the mapping system.

In some embodiments of the invention, the mapping system includes a small number of electrodes which are moved to generate an electrical activity map of tissue near the coronary sinus. Optionally or additionally, the mapping system includes a plurality of electrodes arranged circumferentially and/or axially, so that an area can be mapped axially and/or circumferentially at once. In some embodiments of the invention, the mapping system includes enough electrodes placed over a range of axial and circumferential positions, so that it can simultaneously identify both left atrium pacing location and left ventricle pacing location. Once detected, a suitable apparatus (e.g., size, shape) may be selected, the mapping system may be retracted and/or the apparatus may be advanced over the guiding means. In some embodiments of the invention, the mapping system remains in the body between the apparatus and the wall of the coronary sinus. Optionally, the mapping system, at least a distal end thereof, serves as electrodes, e.g., sensing and/or stimulation electrodes for the apparatus.

Mapping the effect and response of electrical stimulation and identify useful and/or suitable locations for pacing the pacing electrodes (struts) may significantly improve sensing and/or pacing threshold which may allow for using lower energy pacing signals which in turn may increase longevity of the battery(s) of the heart stimulation apparatus.

According to some embodiments of the present invention, one or more of the batteries of the apparatus may be replaced and/or recharged while the apparatus is located and anchored in its determined location in the coronary sinus of the subject. In particular, the battery(s) are detachably connected to the apparatus via a snap-fit element and may be replaced in an intravenous procedure in which an intravenous system may be operated to unlock the snap-fit element, retrieve the old battery and attach a new one.

Replacing the battery(s) of the heart stimulation apparatus while located (deployed) in the coronary sinus may enable battery replacement without removing the apparatus in a relatively simple intravenous procedure involving no major invasive surgical procedures. The battery replacement procedure may be therefore significantly simpler compared to replacement of the pacing unit as may be done by some of the existing methods which may require a surgical procedure and thus may cause bleeding and/or injury of the subject. The risks of replacing the pacing device in terms of injury, bleeding and/or tissue damage may be highly increased when replacing leadless pacing device which are typically embedded in tissue. Moreover, the replacement process of the pacing device may degrade the pacing device itself and may in some cases may even result in a malfunctioning device. Furthermore, replacing the pacing device may result in dislocation of the pacing device which may significantly degrade its pacing and/or sensing functionality and performance.

Replacing the battery(s) which are detachably connected to the heart stimulation device in an intravenous procedure with no surgical procedure may thus significantly reduce complexity, time, and/or effort and may also reduce health and injury risk and/or complications to the subject during and/or after the procedure. Also, battery replacement may avoid the need for an electrophysiologist. Replacing the battery(s) of the stimulation apparatus without removing it and/or dislocating it may also prevent potential dislocation of the apparatus thus avoiding degradation to its pacing and/or sensing performance.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM) , a read-only memory (ROM), an erasable programmable read-only memory (EPROM or Flash memory), a static random access memory (SRAM) , a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer program code comprising computer readable program instructions embodied on a computer readable medium may be transmitted using any appropriate medium, including but not limited to wireless, wire line, optical fiber cable, RF, etc., or any suitable combination of the foregoing.

The computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

The computer readable program instructions for carrying out operations of the present invention may be written in any combination of one or more programming languages, such as, for example, assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

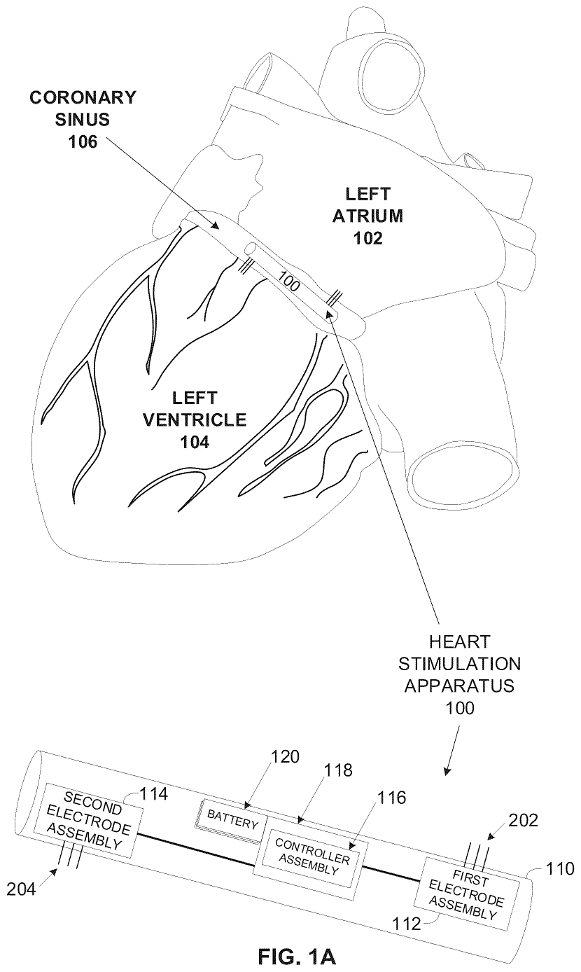

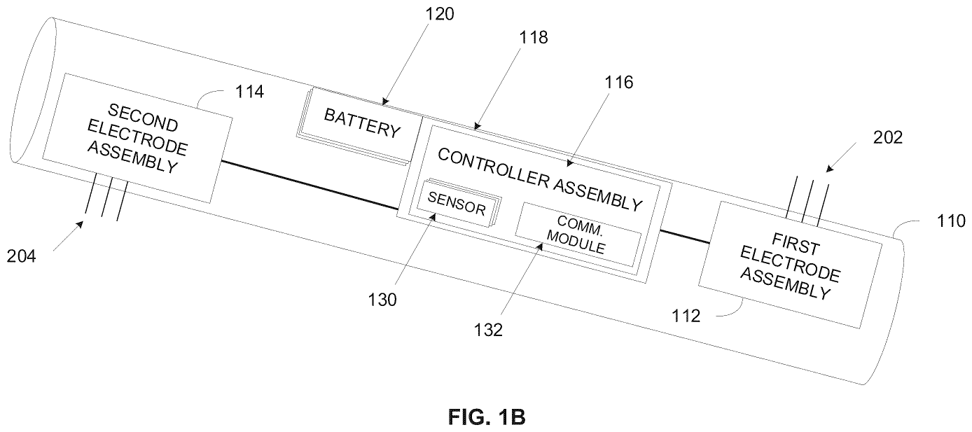

Referring now to the drawings, FIG. 1A and FIG. 1B are a schematic illustrations of an exemplary heart stimulation apparatus designed and configured for deployment in the coronary sinus of a subject for artificially pacing the left atrium and/or the left ventricle of the subject, according to some embodiments of the present invention.

An exemplary apparatus 100 for artificial stimulation of the heart of a subject, for example, a human, a mammal, and/or the like may be configured to be deployed in the coronary sinus 106 of the subject in order to artificially pace the left atrium 102 of the subject and/or the left ventricle 104 of the subject. The artificial stimulation apparatus 100 may be deployed and placed in the coronary sinus 106 for permanent pacing of the subject's heart, e.g., for a long time period (e.g., at least a week, at least a month, at least a year) rather than for temporarily, for example, during medical surgery, treatment, and/or therapy. Power storage of the device may be set up according to an expected duration of use.

The artificial stimulation apparatus 100 may comprise an elongated body 110 configured to be located (placed) in the coronary sinus 106 of the subject. A first electrode assembly 112 which is mechanically coupled to the body 110 may be configured for electrifying the left atrium 102 when the body 110 is located and positioned accordingly in the coronary sinus 106. A second electrode assembly 114 which is also mechanically coupled to the body 110 may be configured for electrifying the left ventricle 104 when the body 110 is located and positioned accordingly in the coronary sinus 106. It is a particular feature of some embodiments of the invention that both the left atrium and left ventricle may be paced simultaneously, due to the apparatus geometry, especially length and electrode orientation. Optionally, the number of electrodes is relatively small (e.g., <5, 3 for each assembly) and oriented over a small angular range, for example, less than 180 (or 100) degrees for each of the first electrode assembly 112 and second electrode assembly 114.

The apparatus 100 may be a single piece device, e.g., forming a single unit rather than a plurality of distinct units deployed at different locations in the subject's blood system and optionally communicatively coupled to each other. In some embodiments, apparatus 100 comprises a plurality of mechanically distinct sections, coupled together by one or more coupling sections, for example, in the form of cabling or other, more flexible sections. For example, the mechanically distinct parts may all include anchoring ability while the coupling sections may not.

However, since the locations for effectively electrifying the (muscle of) left atrium 102 and the (muscle of) left ventricle 104 may be relatively distant from each other, for example, 50 millimeters (mm), the body 110 may have an elongated shape and/or structure having a length of, for example, 40-70 mm thus extending along a substantial section of the coronary sinus 106 between the effective electrification spots. The body 110 may be constructed of one or more materials, and/or coated with such materials, for example, Stainless Steel, Cobalt-Chromium, a polymer, and/or the like.

The first electrode assembly 112 and the second electrode assembly 114 may be mechanically coupled to the body 110 sufficiently distant from each other, for example, 10-100 mm to be effectively oriented, when the body 110 is located in the coronary sinus 106, to extend towards the left atrium 102 and the left ventricle 104 respectively for efficient electrification. In some embodiments of the invention, the orientation of the electrodes can have a relatively low precision, due to the ability to select which electrodes to actually use for electrification, after the implantation. For example, a user may use the electrodes to sense electricity in the heart and based on that send instructions to the apparatus 100 (e.g., using a wireless transmission or via a wired connection of the implantation system), the apparatus circuitry/controller (e.g., 116, noted below) may include a switch selecting which electrodes are electrified.

Stimulating and electrifying the left atrium 102 and/or the left ventricle 104 may significantly improve the artificial pacing of the subject's heart compared to existing pacemakers and artificial stimulation devices which are typically configured and deployed to stimulate the ventricular septum, for example, leadless pacing devices which are capable of pacing only the ventricle. Some pacing schemes may involve two distinct leadless pacing devices, one deployed in the ventricle and the other in the atrium which may communicate and synchronize pacing with each other. However, implanting two separate devices in two different locations may increase risk for complications and may significantly complicate and/or increase the time of the implanting procedure. Moreover, such a two devices deployment may be exposed to increased potential malfunctions since, first, any one of the two devices may fail and, second, communication between the two devices may fail. In addition, battery depletion at the two distinct pacing devices may be different which may require separate and/or early battery replacement for the two devices.

Moreover, since the first electrode assembly 112 and the second electrode assembly 114 are located and oriented to electrify the left atrium 102 and the left ventricle 104 respectively from within the coronary sinus 106, the first electrode assembly 112 and the second electrode assembly 114 may inject electrical signals into the natural pacing signal conduction system delivering pacing signals to the muscles of the left atrium 102 and the left ventricle 104. The electrical signals driven by the electrode assemblies, in particular by the second electrode assembly 114 may therefore induce physiological pacing which may be substantially aligned and/or synchronized with the natural cardiac pacing signal of the heart. This synchronized stimulation may significantly improve the artificial pacing effect and/or reduce damage and/or deterioration to the muscle cells of the left atrium 102 and/or the left ventricle 104.

The apparatus 100 may further include a controller assembly 116 which is electrically coupled to the first electrode assembly 112 and to the second electrode assembly 114. The controller assembly 116 may comprise control logic configured to drive one or more pacing electrical signals to the first electrode assembly 112 and/or to the second electrode assembly 114 and optionally sense electrical signals via the and/or apply pacing logic and timing.

The fact that the heart stimulation apparatus 100 is configured and deployed to pace the left parts of the heart, namely the left atrium 102 and the left ventricle 104 may be advantageous not only in increased efficiency pacing but also in better synchronizing the pacing electrical activity, similar to eft bundle branch pacing (LBBP) and may also reduce deterioration of function of the left ventricle 104. Using the heart stimulation apparatus 100 may even improve HF patients. Moreover, the fact that several electrodes are used for driving the pacing signals may support different pacing configurations which may improve pacing and/or sensing performance which may also result in better longevity of apparatus's battery(s).

The controller assembly 116 may be configured to adjust one or more signal parameters of the pacing electrical signals driven to the first electrode assembly 112 and/or to the second electrode assembly 114, for example, a pacing timing, a frequency, a pulse width, a signal amplitude, a rise time, fall, time, and/or the like. for example, the pacing electrical signals driven by the controller assembly 116 to the first electrode assembly 112 and/or to the second electrode assembly 114 may be characterized by an amplitude in a range of 0.5-5.0 Volt (V) and a pulse with of 0.15-1.5 milliseconds (ms).

Optionally, the controller assembly 116 may be further configured to drive, adjust, and/or inhibit the pacing electrical signal driven to the first electrode assembly 112 and/or to the second electrode assembly 114. For example, controller assembly 116 may sense an electrical signal an calculate a desired activation time and drive a pacing signal to an electrode assembly accordingly. For example, sensing may be based on sensing in the left atrium 102 and/or in the left ventricle 104. For example, the controller assembly 116 may be configured to inhibit the pacing electrical signal intended to be driven to the first electrode assembly 112 for stimulating the left atrium 102 in case a natural cardiac pacing signal is detected the left atrium 102. In another example, the controller assembly 116 may be configured to inhibit the pacing electrical signal intended to be driven to the second electrode assembly 114 for stimulating the left ventricle 104 in case a natural cardiac pacing signal is detected in the left ventricle 104. In another example, the controller assembly 116 may be configured to inhibit the pacing electrical signal intended to be driven to the second electrode assembly 114 for stimulating the left ventricle 104 in case a natural cardiac pacing signal is detected in the left atrium 102. In another example, timing of a pacing signal to the left ventricle 104 depends on a timing of activation in the left atrium 102 and lack of activation in the left ventricle within a time window.

The controller assembly 116 may receive the cardiac pacing signals from the first electrode assembly 112 and/or second electrode assembly 114. Specifically, the controller assembly 116 may receive cardiac pacing signals naturally induced in the left atrium 102 from the first electrode assembly 112 and cardiac pacing signals naturally induced in the left ventricle 104 from the second electrode assembly 114. The assembly may also sense signals from further away in the heart (e.g., global activity or right ventricle activity or right atrium activity), by using electrodes from both assemblies.

The controller assembly 116 may optionally include one or more circuitries, devices, sensors and/or the like for sensing, attenuating, filtering, and/or processing the cardiac pacing signals sensed by the first electrode assembly 112 and/or to the second electrode assembly 114.

Optionally, the controller assembly 116 may comprise one or more sensors 130, for example, motion sensors, for example, an accelerometer, a gyroscope, and/or the like configured and/or operable to capture motion data which may be indicative of motion of the subject. The controller assembly 116 may optionally adjust the pacing electrical signal driven to the first electrode assembly 112 and/or to the second electrode assembly 114 according to the captured motion data. In particular, the controller assembly 116 may analyze the captured motion data to identify one or more motion patterns and adjust the pacing electrical signal accordingly. For example, assuming that, based on analysis of the motion data, the controller assembly 116 identifies a motion pattern indicative of an increased and/or intense physical activity of the subject, for example, running, climbing, and/or the like. In such case, the controller assembly 116 may increase frequency of the pacing electrical signal driven to the first electrode assembly 112 and/or to the second electrode assembly 114 to increase blood circulation in the body of the subject. In example, assuming that, based on analysis of the motion data, the controller assembly 116 identifies a motion pattern indicative of very low physical activity of the subject, for example, sleeping, resting, and/or the like. In such case, the controller assembly 116 may reduce frequency of the pacing electrical signal driven to the first electrode assembly 112 and/or to the second electrode assembly 114 to reduce blood circulation in the body of the subject. In another example, the sensors 130 may comprise one or more impedance sensors configured to capture, measure, and/or monitor one or more measures, indexes, parameters and/or the like in the coronary sinus 106, for example, fluid retention, wall thickness, wall thickness change, and/or the like.

Optionally, the controller assembly 116 may comprise a communication module 132 comprising hardware and/or software elements operable to communicate with one or more remote devices over one or more wireless communication channels and/or networks, for example, Bluetooth (BT), Bluetooth Low Energy (BLE), Wireless Local Area Network (WLAN, e.g., Wi-Fi), and/or the like.

Via the communication interface 132, the controller assembly 116 may communicate, for example, with external equipment, for example, one or more devices, and/or systems configured to monitor, control, calibrate, and/or configure the controller assembly 116 and/or its operation mode. In another example, the controller assembly 116 may communicate via the communication interface 132 with one or more other wireless heart stimulation devices, for example, a pacemaker, an Implantable Cardioverter-Defibrillator (ICD), and/or the like deployed in the body of the subject for stimulating his heart. In another example, the controller assembly 116 may communicate via the communication interface 132 with one or more external defibrillators in order to synchronize with operation of the external defibrillator, for expiable, stop driving pacing electrical signals to the first and/or second electrode assemblies while the defibrillator is operated to stimulate the subject's heart.

Optionally, the communication interface 132 may comprise one or more wired communicating interfaces, channels, and/or ports, for example, a serial port, a Universal Serial Bus (USB) port, a LAN port, and/or the like through which the controller assembly 116 may communicate with one or more other devices, for example, an intravenous delivery system configured and operated to deliver the apparatus 100 to its designated implant location, a programmer used to configured, calibrate and/or test the apparatus 100, and/or the like.

The controller assembly 116 which is also mechanically coupled to the body 110 may comprise a signal generator for generating the pacing signals and may further comprise control logic for controlling, defining, setting and/or adjusting the pacing signals generated by the signal generator and driven to the first electrode assembly 112 and/or to the second electrode assembly 114.

The control logic may be facilitated by hardware, firmware, software and/or a combination thereof. For example, the control logic may be implemented using one or more discrete semiconductor devices, for example, an integrated Circuit (IC), and/or the like. In another example, the control logic may be facilitated using one or more processors, controller, and/or the like capable of executing program instructions stored in one or more memory devices, specifically persistent memory devices such as, for example, ROM, Flash, and/or the like. In another example, the control logic may be implemented using one or more application specific devices, for example, a Field Programmable Gate Array (FPGA), an Application Specific IC (ASIC), and/or the like potentially integrating one or more processors, controller, and/or the like capable of executing program instructions. Naturally, the control logic may be utilized thorough any combination of two or more of these possible implementations.

The control assembly 116 may be encapsulated in a housing 118 designated capsule herein after which may be constructed of one or more materials and/or coated with one or more such materials, for example, Stainless Steel, Cobalt-Chromium, a polymer, and/or the like to provide a sealed environment for the controller assembly 116 contained in it and isolate the controller assembly 116 from the surrounding blood stream while the apparatus 100 is in the subject's body.

The apparatus 100 may comprise one or more batteries 120, for example, a Lithium battery, an alkaline battery, a Zinc-Carbon battery, and/or the like electrically connected to the control assembly 116 for powering the control assembly 116 and its functional elements and/or module, for example, the signal generator, the control logic, and other optional circuits the control assembly 116 may comprise, for example, a sensor, a communication module, and/or the like. The battery(s) 120 may connect to the capsule 118 via one or more terminals for delivering the electrical current to the encapsulated controller assembly 116.

Optionally, one or more of the battery(s) 120 may be rechargeable while in the body of the subject, in particular, while the apparatus 100 is located in the coronary sinus 106. The rechargeable battery(s) 120 may be charged in the body via one or more charging ports of the apparatus 100, which may be disposed, for example, in the rechargeable battery(s) 120 and/or in the capsule 118.

For example, the heart stimulation apparatus 100 may comprise one or more connectors adapted for charging the rechargeable battery(s) 120. For example, a male-female push-pull connector having a certain number, for example, five leads may be used for charging the rechargeable battery(s) 120 where the female connector, for example, may be installed at the apparatus side and the male connector may be deployed in an intravenous system configured and operable for charging rechargeable battery(s).

Such an intravenous system may be operated to electrically connect to one or more of the charging ports of the apparatus 100 for a certain charging time in order to charge the rechargeable battery(s) 120. In some embodiments of the invention, the apparatus 100 may continue operating to stimulate the left atrium 102 and/or the left ventricle 104 with the pacing signal even while the rechargeable battery(s) 120 is being charged.

In some embodiments of the invention, charging is wireless. For example, the terminals may be replaced by a power receiving circuit, such as a coil and a catheter with a transmitting coil advanced to near the terminals. Optionally or additionally, charging is via an external charger.

Optionally or additionally to charging, a wireless transmitter/receiver may be used to transmit and/or receive data (e.g., logs) and instructions (e.g., settings).

Optionally, one or more of the battery(s) 120 may be detachably attached to the capsule 118 via one or more releasable snap-fit elements as described herein after in detail. A n intravenous system configured and operable for retrieving detachable batteries may be operated to retrieve one or more of the detachable battery(s) 120 and typically replace them with new batteries.

Optionally, the heart stimulation apparatus 100 may comprise one or more devices, circuitries, and/or elements configured to convert kinetic energy to electrical charge for charging one or more of the batteries 120. As such, while the subject is in motion, the kinetic energy conversion devices included in the apparatus 100 may convert kinetic energy induced by the subject's bodily motion to produce electrical energy which may be driven, optionally via one or more charging circuits, to charge the battery(s) 120.

Optionally, the heart stimulation apparatus 100 may comprise one or more energy harvesting devices, circuitries, and/or elements configured to capture radiant energy from ambient electromagnetic waves and convert this energy to an electrical charge for charging one or more of the batteries 120. The harvesting devices may intercept electromagnetic radiation emitted, for example, by one or more extremal devices attached to the subject's body.

The first electrode assembly 112 and/or the second electrode assembly 114 may comprise penetrating electrodes designed, adapted and deployed to penetrate through the walls of the coronary sinus 106 into the muscle tissue of the left atrium 102 and left ventricle 104 respectively.

For example, each of the first electrode assembly 112 and/or the second electrode assembly 114 may each comprise a plurality of electrodes, optionally penetrating electrodes, optionally sharpened, optionally in the form of micro-needle struts (studs) (e.g., elongated narrow sharpened semi-rigid or rigid elements) extending from the body 110 for penetrating through a wall of the coronary sinus 106 when the apparatus 100, specifically the body 110 is located in the coronary sinus 106 and securely anchored in place. For example, the first electrode assembly 112 may comprise a plurality of atrial struts 202 extending from the body 110 through a wall of the coronary sinus 106 for penetrating into muscle tissue of the left atrium 102 when the apparatus 100 is located in the coronary sinus 106. In another example, the second electrode assembly 114 may comprise a plurality of ventricular struts 204 extending from the body 110 through a wall of the coronary sinus 106 for penetrating into muscle tissue of the left ventricle 104 when the apparatus 100 is located in the coronary sinus 106.

The plurality of atrial struts 202 and ventricular struts 204 may be shaped and sized to penetrate through the wall of the coronary sinus 106 into the muscle tissue of the left atrium 102 and left ventricle 104 respectively. However, a length of the atrial struts 202 and ventricular struts 204 may be limited to a certain length to prevent them from penetrating through the endocardium of the left atrium 102 and/or the left ventricle 104. For example, an average thickness of the left atrium 102 may be approximately 4 mm. The atrial struts 202 may be therefore limited to a length of, for example, 2-5 mm which does not reach the endocardium of the left atrium 102. In another example, an average thickness of the left ventricle 104 may be approximately 20 mm. The ventricular struts 204 may be therefore limited to a length of, for example, 12-18 mm which does not reach the endocardium of the left ventricle 104.

The atrial struts 202 and ventricular struts 204 may be produced and/or constructed of one or more materials, alloys, and/or metals optimized for electrical signals delivery to ensure efficient sensing and/or stimulation, for example, Platinum-Iridium (Pt/Ir). The atrial struts 202 and/or ventricular struts 204 may be designed and/or constructed as micro-electrodes having a diameter adapted for efficient penetration into the muscle tissue of the left atrium 102 and the left ventricle 104.

Optionally one or more of the atrial struts 202 and/or ventricular struts 204 may have a varying diameter along their longitudinal axis such that the diameter may be larger at their base where they connect to the electrode assemblies and smaller at their tip.

Optionally, one or more of the atrial struts 202 and/or ventricular struts 204 may be coated using one or more materials, for example, steroid-eluting materials, oxide coating, Rapamycin like compounds, and/or the like.

Moreover, one or more of the atrial struts 202 and/or ventricular struts 204 may be adapted, constructed, and/or shaped to have a straight structure and/or an at least partially curved structure such that they may penetrate the muscle tissue straight and/or in at least partially curved angle respectively. Straight struts may be easier to place and remove, while curved struts may apply more pressure on the muscle tissue which may improve contact with the muscle tissue which may improve sensing and/or electrifying functionality.

Each of the plurality of atrial struts 202 and ventricular struts 204 may comprise one or more electrical leads (e.g., with an exposed contact area) for delivering the pacing electrical signal. The electrode assemblies 112 and/or 114 may employ one or more architectures, deployments and/or wiring for delivering the pacing electrical signal to the muscle tissue of the left atrium 102 and/or the left ventricle 104 via their respective struts 202 and 204.

For example, one or more of the struts atrial struts 202 and/or ventricular struts 204 may be single-terminal struts comprising a single lead providing a single polarity (e.g., cathode and/or anode) of the respective electrode assemblies 112 and/or 114. In such case, the electrical signal driven to the first electrode assembly 112 may go through the muscle tissue of the left atrium 102 between two or more atrial struts 202 and the electrical signal driven to the second electrode assembly 114 may go through the muscle tissue of the left ventricle 104 between two or more ventricular struts 204.

In another example, one or more of the atrial struts 202 and/or ventricular struts 204 may be multi-terminal (multi-polar) struts comprising two or more leads of which some may serve as the cathode and others as the anode of the respective electrode assemblies 112 and/or 114, for any given pacing pulse section. In such case, the electrical signal driven to the first electrode assembly 112 may go through the muscle tissue of the left atrium 102 between different terminals of the same atrial strut(s) 202 and/or between terminals of one or more different atrial struts 202. Similarly, the electrical signal driven to the second electrode assembly 114 may go through the muscle tissue of the left ventricle 104 between different terminals of the same ventricular strut(s) 204 and/or between terminals of one or more different ventricular struts 204.

The atrial struts 202 and ventricular struts 204 may be shaped, adapted, and/or constructed according to one or more designs, and/or structures to achieve both efficient penetration to muscle tissue and efficient sensing and/or electrifying the muscle tissue. For example, one or more of the atrial struts 202 and/or ventricular struts 204 adapted for sensing and/or stimulation may be shaped to have a hollow pipe shape having a length within a certain range, for example, 0.5-5 mm, 0.7-4 mm and/or 1-3 mm with a certain tip, for example, a round tip. In another example, one or more of the atrial struts 202 and/or ventricular struts 204 adapted for sensing may be shaped to have ball shape with a diameter selected from a certain range, for example, 0.2-8 mm, 0.7-5 mm, and/or 1-3 mm.

Optionally, one or more of the atrial struts 202 and/or ventricular struts 204 may be shaped, constructed, and/or designed to have a step change with an increased diameter at its proximal end, for example, at their base wherein they connect to the electrode assemblies to prevent over penetration into the muscle tissue of the left atrium 102 and/or the left ventricle 104 respectively.

The controller assembly 116 may optionally comprise one or more circuits, for example, a switch, a gate, and/or the like for selecting specific struts of the plurality of atrial struts 202 and/or ventricular struts 204 to electrify such that pacing electrical signal may be driven only to selected struts.

Moreover, one or more of the atrial struts 202 and/or ventricular struts 204 may be used to sense, through their leads, the cardiac pacing signals naturally induced to pace the left atrium 102 and/or the left ventricle 104 respectively. Circuitry assembly 116 may be configured to selectively use a same electrode for sensing and for stimulation, and/or for permanently using an electrode for only sensing or stimulating.

One or more implementations, modes, and/or techniques may be applied to dispose the electrical leads in the atrial struts 202 and ventricular struts 204. In some embodiments of the invention, the electrical leads of the struts are electrically insulated from each other and optionally from the struts themselves which may be made of one or more conductive material(s).

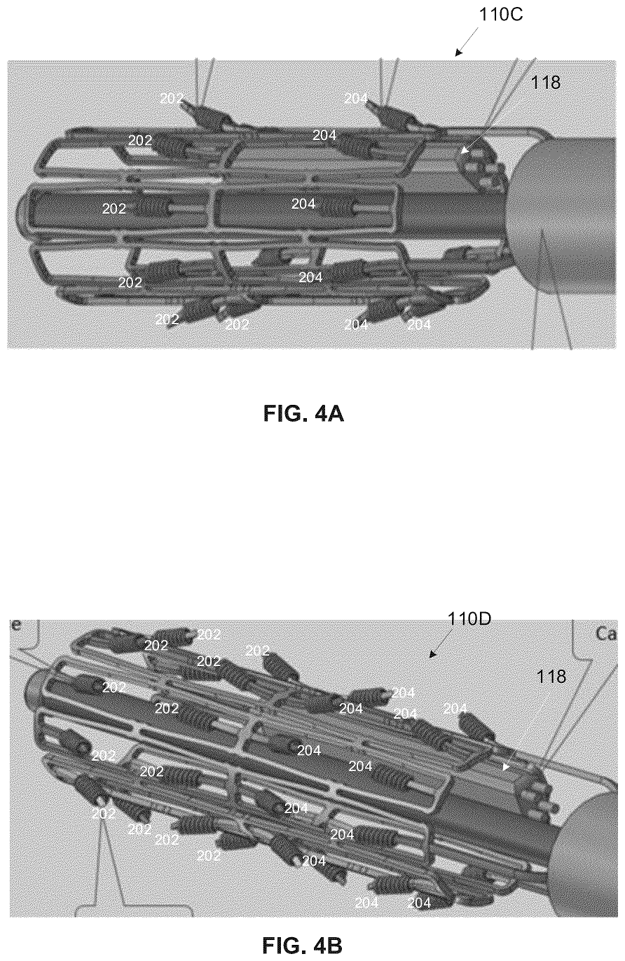

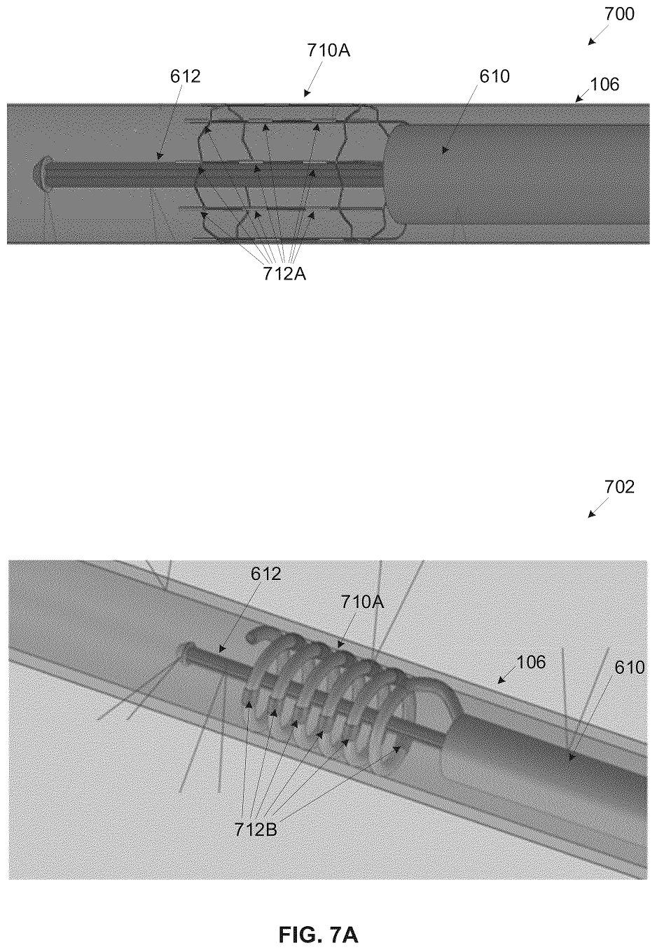

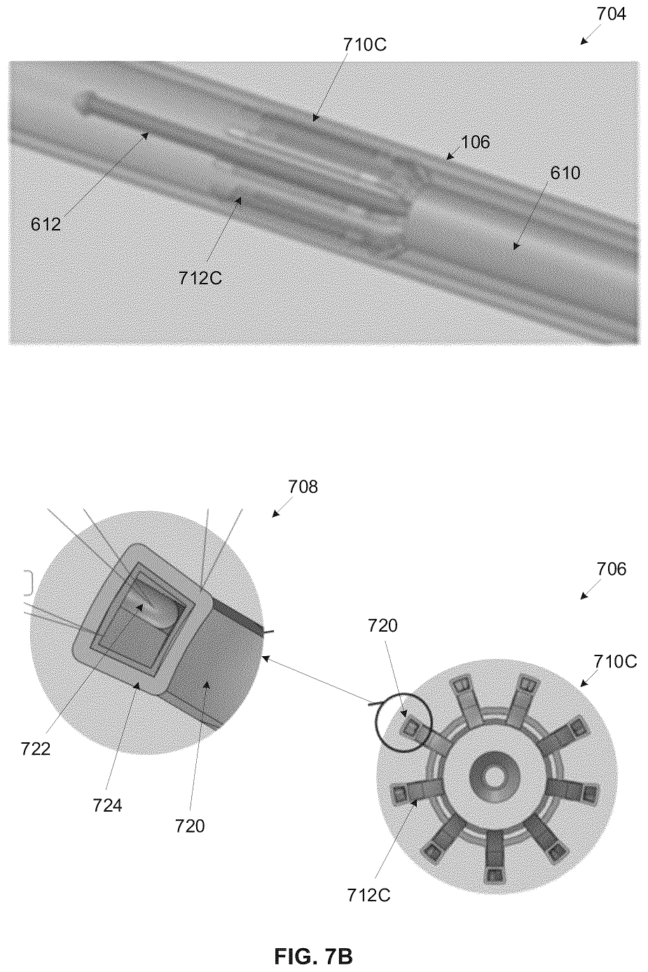

For example, one or more electrical leads may be disposed externally on one or more of the atrial struts 202 and ventricular struts 204, for example, wrapped, stretched, adhered and/or deposited thereon (e.g., FIG. 4B). In another example, one or more electrical leads may extend from an interior bore (e.g., FIG. 7B) of one or more of the atrial struts 202 and ventricular struts 204. For example, assuming one or more leads of one or more of the atrial struts 202 and/or ventricular struts 204 are multi-terminal struts each comprising a two electrical leads. In such case, a first electrical lead may be deposed externally on the strut and a second electrical lead may extend from an interior bore of the strut such that they are electrically isolated from one another. In another example, two or more electrical leads may be disposed externally on one or more of the atrial struts 202 and/or ventricular struts 204 where each electrical lead is covered by isolation material up to its distal end which is exposed and comes in physical contact with the muscle tissue of the atrium 102 and/or ventricle 104 respectively. In case of single-terminal atrial struts 202 and/or ventricular struts 204, a single electrical lead may be disposed externally and/or internally in the respective strut.

Optionally, one or more of the plurality of struts, e.g., the atrial struts 202 and/or the ventricular struts 204 may be shaped to have a structure to support effective anchoring of the body 110A in place when located in the coronary sinus 106. For example, one or more of the atrial struts 202 and/or ventricular struts 204 may have a bent end, for example, arched, angled, hooked and/or the like such that they may effectively attach to the wall of the coronary sinus 106 and anchor the apparatus 100, specifically the body 110A in place.

Optionally, the apparatus 100 may comprise one or more anchoring elements mechanically coupled to the body 110 for anchoring the body 100 in place when located in the coronary sinus 106. For example, the apparatus 100 may comprise multiple anchoring elements, for example, elements currently shown as electrodes but not including electrodes therewith), for example, 3, 4, 6, or more which are mechanically coupled to the body 110 at one end and bent (e.g., arched, angled, hooked) at their other end, in particular bent opposite the blood stream direction. The anchoring elements may extend from the body 110 perpendicularly and/or at a significantly obtuse angle with respect to the longitudinal axis of the body 110 such that when the body 110 is located (placed) in the coronary sinus 106, the anchoring elements may penetrate through the walls of the coronary sinus 106 and sink into tissue of the coronary sinus 106 and/or tissue of surrounding organs, for example, a muscle, thereby anchoring the body 110 in place.

The apparatus 100 may be designed, constructed, and/or fabricated according to a plurality of designs, architectures, and/or structures to ensure that when the apparatus 100 is located in the coronary sinus 106, the body 110 is effectively, reliably and robustly anchored in its designated place to effectively pace the left atrium 102 and/or the left ventricle 104 without degrading the flow of blood through the coronary sinus 106, and with minimal and potentially no risk or damage to the subject's internal organs and blood vessels.

Optionally, the body 110 may be constructed of a plurality of separate segments which are mechanically coupled to each other via one or more coupling elements. The coupling element(s), comprising for example, a string, a coil, a flexible tube, a bar and/or a thread may be flexible (e.g., at least 5 times as flexible as each separate segment) and/or at least partially elastic to enable some flexibility and freedom of movement (e.g., bending and/or rotation and/or axial extension/shrinking) between at least some of the segments for effective maneuverability and positioning of the body 110 in a determined location in the coronary sinus 106. However, while comprising multiple segments, the apparatus 100 is a single device which is delivered to and located in the coronary sinus 106 as a single piece with no separate/multiple deliveries and/or placements in the coronary sinus 106 or other blood vessels.

The plurality of segments of the body 110 may be designed shaped and located in the coronary sinus 106 with respect to the atrium 102 and/or the left ventricle 104 to position and orient the first and second electrode assemblies 112 and 114, attached to these segments, to extend towards the left atrium 102 and/or the left ventricle 104 respectively. As such, when the body 110 is anchored in its designated location in the coronary sinus 106, the atrial struts 202 and the ventricular struts 204 may penetrate through the wall of the coronary sinus 106 and penetrate into the muscle tissue of the left atrium 102 and left ventricle 104 respectively optionally at precise and accurate location selected for effective electrical signal simulation and/or sensing.

In some embodiments of the invention, the segments are moved relative to each other during delivery, to provide proper placement of electrodes thereof. Optionally or additionally, such flexibility is used after implantation, for example, to better to conform to changes in the coronary sinus geometry during a cardiac cycle.

While some specific embodiments of the apparatus 100 are described herein, other designs and structures may become apparent to a person skilled in the art as covering the same teachings and the described embodiments should therefore not be construed as necessarily limiting. Some alternative structures are described below and which may use any of the features described for apparatus 100.

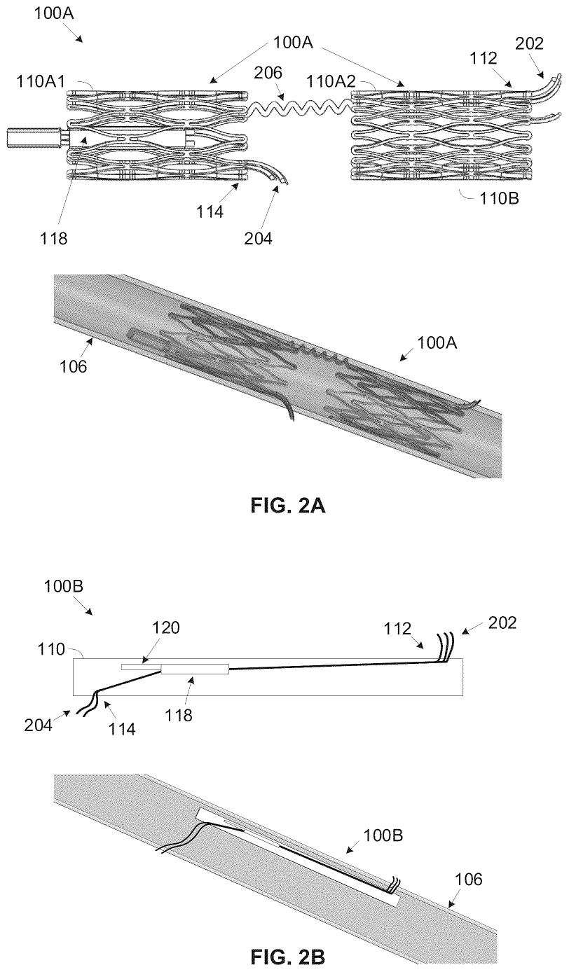

Reference is now made to FIG. 2A and FIG. 2B, which are schematic illustrations of exemplary embodiments of a heart stimulation apparatus designed and configured for deployment in the coronary sinus of a subject for artificially pacing the left atrium and/or the left ventricle of the subject, according to some embodiments of the present invention.

As seen in FIG. 2A, an exemplary heart stimulation apparatus 100A such as the heart stimulation apparatus 100 may be constructed, shaped, and/or adapted to have a cylindrical structure with flow-lumen therein, for example, at least partially as a stent. As such, the heart stimulation apparatus 100A, in particular, a body 110A such as the elongated body 110 of the heart stimulation apparatus 100A may have a stent-like structure. For example, the heart stimulation apparatus 100A may consist of a flexible, thin-strut nitinol stent, crimped onto a customized delivery catheter and specialized wires able to safely deliver electrical impulses via the blood vessels route.

By stent-like is meant that the segments, for example, two segments, of the heart stimulation apparatus 100A are generally tubular shaped frames which expand to anchor in a blood vessel. However, unlike standard stents, there is no need for the stent shaped segments to support the blood vessel against collapsing. Also, the expansion force can be reduced if the electrode assemblies or other anchor elements act to anchor apparatus 100.

So, while the apparatus 100 does not necessarily function as a stent in the sense that it supports the walls of the coronary sinus 106 to keep it open and improve blood flow, the stent-like body 110A may be located in the coronary sinus 106 in an expanded state same as a stent such that it closely engages the inner walls of the coronary sinus 106. Optionally, deployment uses stent-delivery technologies, such as self-expansion and/or balloon expansion.

The stent-like body 110A having thin walls and a large lumen may have very little and typically negligible impact on the flow of blood through the coronary sinus 106. The stent-like body 110A, which may optionally be segmented to include, for example, two independent segments connected to each other via some mechanical and/or electrical coupling elements, may be designed to achieve a minimal radial strength required for sufficient contact with the inner walls of the coronary sinus 106 while allowing efficient and smooth delivery through blood vessels, typically ≤10 mm, and sharp bends. The stent-like body 110A may be constructed as a self-expandable NiTi braided stent.

The stent-like configuration of the heart stimulation apparatus 100A may significantly simplify delivery and/or removal of the heart stimulation apparatus 100A.

The stent-like configuration of the heart stimulation apparatus 100A may be used only for anchoring the heart stimulation apparatus 100A in place without applying force on the walls of the coronary sinus 106. The stent-like body 110A may be therefore thinner and/or weaker than standard stents, especially arterial stents.

Exemplary dimensions for the length of the stent-like body 110A may be in a range of 8-17 mm, 9-16 mm, and/or 10-15 mm. The length of the stent-like body 110A may be as minimal as possible, while still supporting re-sheathing prior to full release.

Exemplary length for the stent-like body 110A may be in a range of 3-10 mm, 4-9 mm, 5-8 mm, and/or the like in the folded state (compressed, retracted) and 12-18 mm, 13-17 mm, and/or 14-16 mm in the expanded state. The dimeters of each of the segments may be uniform or not (e.g., tapering) and/or may be the same or different (noting that the coronary sinus tapers along its length.

The apparatus 100A may comprise a capsule such as the capsule 118 mechanically coupled to the body 110A, in particular to one or more of the inner walls and/or surfaces of the body 110A. One or more bonding means may be used for mechanically coupling (bonding) the capsule 118 to the body 110A.

One or more batteries such as the battery 120 may be attached to the capsule 118 (or provided therein) for powering a controller assembly such as the controller assembly 116 encapsulated in the capsule 118. The capsule 118 and the attached battery(s) 120 may have a very small profile, specifically in the plane perpendicular to the longitudinal axis of the coronary sinus 106, potentially not significantly affecting the flow of blood through the coronary sinus 106. A potential advantage of having two batteries (one possibly permanently attached) is that circuitry 116 can be powered even while the batter is being replaced.

Each of the electrode assemblies 112 and 114 may electrically connect to the controller assembly 116 via one or more leads disposed in the elements composing the stent-like body 110A, for example, wires, coated conductors, deposited conductors, rings, connectors and/or links. Leads of a plurality of atrial struts such as the atrial struts 202 of the first electrode assembly 112 may electrically connect to the leads coming in from the controller assembly 116 to receive electrical pacing signals driven from the controller assembly 116 and deliver them to muscle tissue of the left atrium 102. Similarly, leads of a plurality of ventricular struts such as the ventricular struts 204 of the second electrode assembly 114 may electrically connect to the leads coming in from the controller assembly 116 to receive electrical pacing signals driven from the controller assembly 116 and deliver them to muscle tissue of the left ventricle 104.

The number of atrial struts 202 and ventricular struts 204 may be, for example, in a range of two to eight. For example, the electrode assembly 112 may consist two atrial struts 202 while the electrode assembly 114 may consist three ventricular struts 204.

Optionally, the number of atrial struts 202 and/or ventricular struts 204 may be selected, defined, and/or set according to the electrical configuration, functionality, and/or structure of the struts. For example, assuming the atrial struts 202 and/or ventricular struts 204 are single polar struts capable of delivering only a single lead, for example, signal and/or return (ground), the number of atrial struts 202 and/or ventricular struts 204 may be increased. In another example, assuming the atrial struts 202 and/or ventricular struts 204 are bi-polar and/or multi-polar struts capable of delivering multiple leads, for example, both signal and return (ground), the number of atrial struts 202 and/or ventricular struts 204 may be reduced.

As noted above, a switch (e.g., in circuitry 116) may be provided to switch power to particular electrodes to be electrified and/or sensed. Optionally, the switch is set using a transmission from outside, for example, using magnetic relays or a RF.

As seen, the atrial struts 202 and the ventricular struts 204 of the first and second electrode assemblies 112 and 114 respectively may be shaped and positioned to penetrate through the wall of the coronary sinus 106 when the stent-like body 110A is located (placed) in its expanded state in the coronary sinus 106.

The body 110A may be anchored in place using one or more dedicated anchoring elements separate from the atrial struts 202 and the ventricular struts 204, for example, an anchor strut, and/or an anchor bar which optionally penetrate through the walls of the coronary sinus 106 and imbed in the coronary sinus 106 and/or in other tissue beyond the walls of the coronary sinus 106. However, optionally at least some of the atrial struts 202 and the ventricular struts 204 may serve as the anchoring elements to anchor the apparatus 100A, specifically, the expanded stent-like body 110A in place.

Optionally, the body 110A is constructed of a plurality of separate segments, for example, two segments 110A 1 and 110A 2 mechanically coupled to each other via one or more mechanical coupling elements, for example, a coil 206 which may be at least partially elastic.

The mechanical coupling elements may be further adapted to route and/or deliver electrical signals between the two segments 110A 1 and 110A 2. For example, assuming the capsule 118 is deployed in segment 110A1, the mechanical coupling elements may be adapted to deliver electrifying electrical signals to segment 110A 2 and/or to receive sensing electrical signals from segment 110A 2. As seen in FIG. 2A, an exemplary heart stimulation apparatus 100B such as the heart stimulation apparatus 100 may be constructed as an elongated body 110B constructed such that when located in the coronary sinus 106 extends between the left atrium 102 and the left ventricle 104, the first electrode assembly 112, specifically the atrial struts 202 may be oriented towards the left atrium 102 and the second electrode assembly 114, specifically the ventricular struts 204 may be oriented towards the left ventricle 104.

As described for the exemplary body 110A of apparatus embodiment 100A, the body 110B may be anchored in place using one or more dedicated anchoring elements and/or by one or more of the atrial struts 202 and/or the ventricular struts 204.

Since the left atrium 102 and the left ventricle 102 may be at least somewhat radially offset from each other around the longitudinal axis of the coronary sinus 106, the atrial struts 202 may be offset from the ventricular struts 204 in a circumferential direction relative to the longitudinal axis of the body 110. In other words, the atrial struts 202 may extend from the body 110 in first radial direction and the ventricular struts 204 may extend from the body in a second radial direction where the first direction is offset from the second radial direction to orient the atrial struts 202 to extend towards the left atrium 102 and orient the ventricular struts 204 towards the left ventricle 104 when the body 110 is located in the coronary sinus 106.

Such orientation of the atrial struts 202 and the ventricular struts 204 may be used to position and orient the atrial struts 202 to penetrate the muscle tissue of the left atrium 102 while orienting the ventricular struts 204 to penetrate the muscle tissue of the left ventricle 104 when the body 110 is located in its designated location in the coronary sinus 106.

One or both electrode assemblies may include struts which extend over a range of radial directions, this range defining a (first, second) circumferential sector. This may be beneficial, for example, if mapping and/or delivery are imperfect and during a calibration or use stage, selected electrodes at selected directions within the sector are chosen for use for electrification and/or sensing. Distribution of the atrial struts 202 over the circumferential sector may increase probability of at least some of atrial struts 202 to penetrate the muscle tissue of the left atrium 102 in location(s) desired for artificial stimulation and pacing of the left atrium 102 when the body 110 is located in the coronary sinus 106.

Similarly, desired penetration of the ventricular struts 204 into the left ventricle 104 may be assisted by the ventricular struts 204 being distributed in a plurality of second radial directions covering second circumferential sector around the longitudinal axis of the body 110. Distribution of the ventricular struts 204 over the second circumferential sector may increase probability that at least some of ventricular struts 204 penetrate the muscle tissue of the left ventricle 104 in location(s) desired for artificial stimulation and pacing of the left ventricle 104 when the body 110 is located in the coronary sinus 106.

Moreover, the first circumferential sector may be offset from the second circumferential sector to orient the plurality of atrial struts 202 to extend towards the left atrium 102 and orient the plurality of ventricular struts 204 to extend towards the left ventricle 104 when the body 110 is located in the coronary sinus 106.

Optionally, the atrial struts 202 and/or the ventricular struts 204 may be distributed over the entire perimeter of the body portion 110 meaning that the first circumferential sector and/or the second circumferential sector may encompass a large sector of the body portion, for example, a sector in a range of 60-360 degrees, 120-360 degrees, and/or the like.

The atrial struts 202 and/or the ventricular struts 204 may be may be spaced from each other across the respective sector, i.e. the first sector or the second sector respectively. This spacing of the atrial struts 202 and/or the ventricular struts 204 may be uniform or it may be non-uniform, for example, higher densities in directions corresponding to more likely pacing/sensing regions and/or regions where a higher resolution of location is desired. The atrial struts 202 and/or the ventricular struts 204 may typically point out in a radial direction. However, optionally and/or alternatively, the one or more of the atrial struts 202 and/or one or more of the ventricular struts 204 may extend out in an angle with respect to a longitudinal axis of the body 110.

The atrial struts 202 and the ventricular struts 204 may be oriented to extend from the body 110 in one or more axial directions with respect to the longitudinal axis of the body 110. The axial direction orientation of the atrial struts 202 and/or the ventricular struts 204 may affect the physical contact of the atrial struts 202 and/or the ventricular struts 204 with the muscle tissue of the left atrium 102 and/or the left ventricle 104 respectively potentially affecting the arterial pacing by the pacing electrical signals driven through the atrial struts 202 and/or the ventricular struts 204. Moreover, in case the atrial struts 202 and/or the ventricular struts 204 serve as anchoring elements, the axial direction orientation of the atrial struts 202 and/or the ventricular struts 204 may affect, for example, strength, efficiency, robustness and/or reliability of attachment of the body 110 to the coronary sinus 106 in the designated location.

For example, the atrial struts 202 and the ventricular struts 204 may be disposed to extend from the body 110 in a common axial direction with respect to the longitudinal axis of the body 110. In another example, the atrial struts 202 and/or the ventricular struts 204 may be oriented to extend in a plurality of directions relative to the axis, e.g., at a plurality of angles including a circumferential component and/or including an axial component. For example, the direction may define oblique and/or acute angles with body 110. These directions may be such that each of the atrial struts 202 and/or the ventricular struts 204 may be disposed to extend in one of the plurality of directions with respect to the longitudinal axis of the body 110.

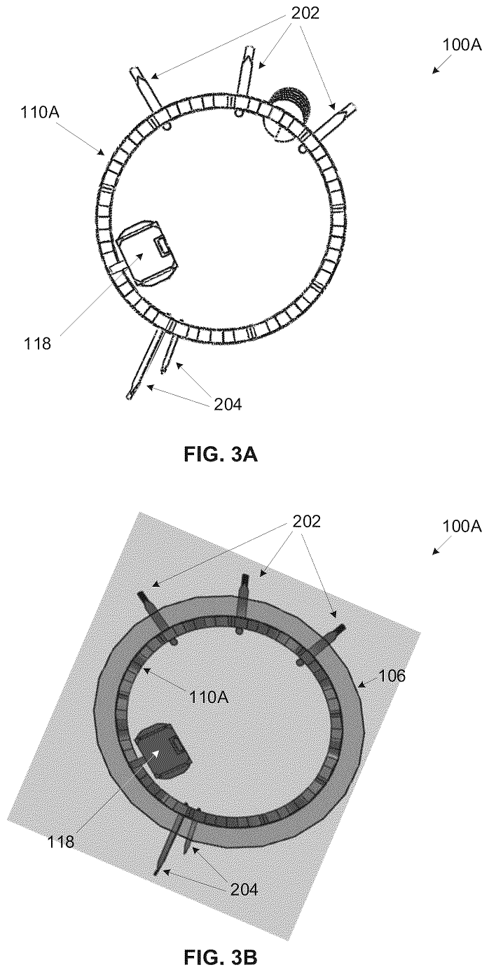

Reference is now made to FIG. 3A and FIG. 3B, which are schematic illustrations of a front view of an exemplary heart stimulation apparatus designed and configured for deployment in the coronary sinus of a subject for artificially pacing the left atrium and/or the left ventricle of the subject, according to some embodiments of the present invention.

As seen in FIG. 3A, an exemplary heart stimulation apparatus such as the heart stimulation apparatus 100A comprising a stent-like body such as the body 110A and a capsule such as the capsule 118 containing a controller assembly such the controller assembly 116 powered by one or more batteries such as the battery 120 attached to the capsule 118 via one or more electrical terminals.

The apparatus 100A further comprises a first electrode assembly such as the first electrode assembly 112 comprising a plurality of atrial struts such as the atrial struts 202 extending outwards from the body 110A, and a second electrode assembly such as the second electrode assembly 114 comprising a plurality of ventricular struts such as the ventricular struts 204 extending outwards from the body 110A.

As seen, the plurality of atrial struts 202 may be radially offset with respect to the ventricular struts 204 according to the radial offset between the left atrium 102 and the left ventricle 104 with respect to the coronary sinus 106.

Moreover, the plurality of atrial struts 202 may be distributed in a plurality of first angles covering a first circumferential sector while the plurality of ventricular struts 204 may be distributed in a plurality of second angles covering a second circumferential sector. It is noted that not all struts (within an assembly and/or between assemblies) need be the same length. For example, all the atrial struts 202 are shown as being the same length, while ventricular struts 204 have different lengths from each other and from struts 202.

As seen in FIG. 3B, when the apparatus 100A is located in the coronary sinus 106, the atrial struts 202 may penetrate the wall of the coronary sinus 106 at the first circumferential sector around the longitudinal (axial) axis of the body 110A into the left atrium 102 while the ventricular struts 204 may penetrate the wall of the coronary sinus 106 at the second circumferential sector around the longitudinal axis of the body 110A into the left ventricle 104.

Reference is now made to FIG. 4A and FIG. 4B, which are schematic illustrations of exemplary deployments of struts extending from a heart stimulation apparatus for electrifying the left atrium and/or the left ventricle of a subject, according to some embodiments of the present invention.

As seen in FIG. 4A and FIG. 4B, a plurality of atrial struts such as the atrial struts 202 and a plurality of ventricular struts such as the ventricular struts 204 may be disposed over a stent-like body such the body 110A of a heart stimulation apparatus such as the heart stimulation apparatus 100A. Each of the atrial struts 202 and the ventricular struts 204 may comprise a lead for electrifying the left atrium 102 and/or the left ventricle 104 respectively. As seen, the atrial struts 202 and the ventricular struts 204 may be disposed to cover the entire circumference of the body 202, i.e., the first and second circumferential sectors may be 360 degree sectors

As seen in FIG. 4A presenting an optional embodiment of the apparatus 100A, the atrial struts 202 and the ventricular struts 204 are disposed to extend from the body 110A in a common axial direction with respect to the longitudinal axis of the body 110A. In another embodiment of the apparatus 100A illustrated in FIG. 4B, the atrial struts 202 and the ventricular struts 204 may be oriented to extend in a different axial directions from the body 110. For example, the atrial struts 202 may extend in a substantially first axial angle with respect to the longitudinal axis of the body 110A while the ventricular struts 204 may extend in a substantially second axial angle with respect to the longitudinal axis of the body 110A.

FIG. 4A shows body 110C extending out of a delivery tube, optionally held stable by one or more holders. It is noted that in an apparatus such as shown in FIG. 2A, a first segment can extend out of the delivery system, as shown in FIG. 4B, while a second segment remains within delivery tube. For example, after implantation of the first, distal, segment, the delivery system may be positioned to deliver the proximal segment at a desired location.

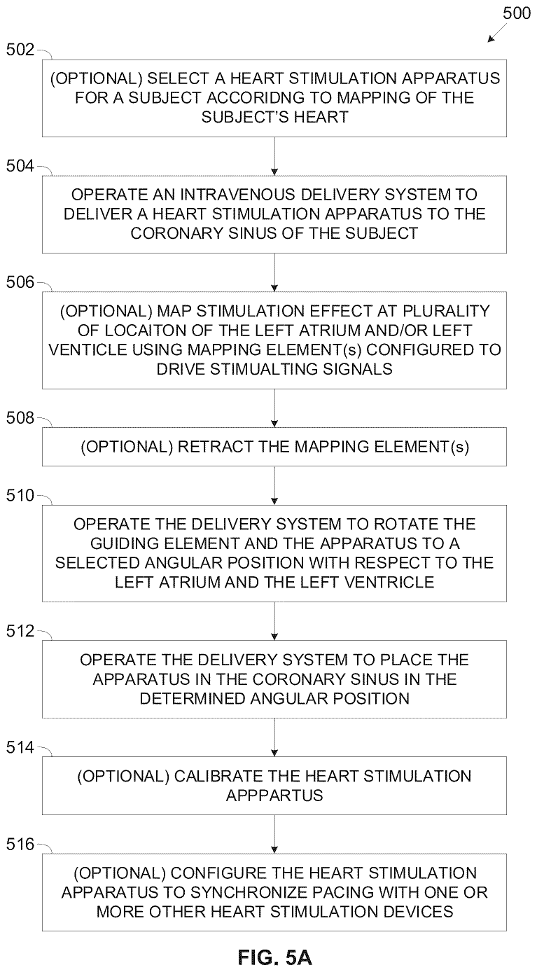

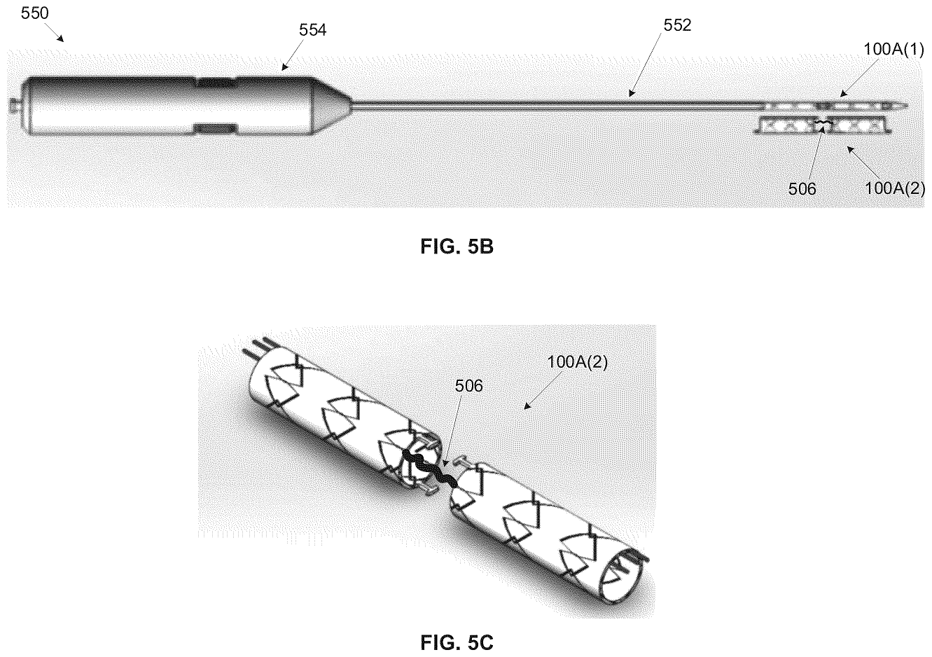

Reference is now made to FIG. 5A, which is a flowchart of an exemplary process for placing a heart stimulation apparatus in the coronary sinus of a subject, according to some embodiments of the present invention.

An exemplary process 500 may be executed to implant a heart stimulation apparatus such as the apparatus 100 in the coronary sinus 106 of a subject in a position and orientation (e.g., angular position) selected for effective pacing and/or sensing of the pacing electrical signals induced to stimulate the subject's heart.

The apparatus 100 may be delivered to the coronary sinus 106 and positioned in it using one or more intravenous delivery systems inserted via one or more routes through the venous system of the subject using one or more techniques, and/or approaches as known in the art for delivering pacemakers. For example, the intravenous delivery systems may employ one or more trans femoral based techniques for deploying arterial/venous access and externalized pacing wires at the coronary sinus. In another example, the intravenous delivery systems may employ one or more trans jugular techniques for delivering and deploying the apparatus 100 may be delivered and positioned at the coronary sinus 106. In another example, the apparatus 100 may be delivered and positioned at the coronary sinus 106 using subclavian approach. Such minimal invasive approach may allow pacing and monitoring during procedures without leads implanted in the heart.

For example, body 110 may be self-expanding and delivered using a delivery tube, which may be retracted while one or more holders hold the body 110 in place. Standard methods may be used to reach the coronary sinus. For example, the intravenous delivery system may be operated, typically under fluoroscopy configured to identify one radio opaque markers marked on the delivery system, and/or other imaging technologies, to advance through an introducer into the right atrium of the subject and after deflection may be rotated towards the coronary sinus ostium. Once it crosses the coronary sinus ostium, the intravenous delivery system may be advanced into the coronary sinus body while releasing the deflection.

In particular, the intravenous delivery system may be operated to position the apparatus 100 in a determined location and position the body 110 in a selected angular position to orient the plurality of atrial struts 202 to extend towards the left atrium 102 and orient the plurality of ventricular struts 204 to extend towards the left ventricle 104.

Once the apparatus 100 is properly positioned in the coronary sinus 106 in its determined location according to the selected angular position, the intravenous delivery system may be operated to anchor the apparatus 100 in place. For example, in case of an apparatus such as the apparatus 100A comprising a stent-like body such as the body 110A, the body 110A may be expanded (e.g., self-expanded by release for a confining over-tube or delivered using an expanding balloon) to its expanded state such that the body 110A may contact at least partially the walls of the coronary sinus 106 and the atrial struts 202, the ventricular struts 204 and/or the dedicated anchoring elements may penetrate through the walls of the coronary sinus 106 to anchor the body 100A in place.

As shown at 502, the process 500 starts with an optional step of selecting the heart stimulation apparatus 100 according to mapping of the heart of the subject and the hearts structure and/or geometry.

The mapping may be done using one or more imaging equipment (e.g., sensor, device, system, platform, etc.) configured to image internal organs of the subject, for example, computerized tomography (CT), Ultrasound imaging (sonography), X-Ray, and/or the like.

The heart stimulation apparatus 100 may be selected, adapted, adjusted and/or fitted according to the mapping of one or more physical features identified for the subject based on analysis of the imaging data captured by the imaging equipment, for example, size of the coronary sinus (e.g. length, diameter, wall thickness, etc.), a relative location (e.g. distance, angle, etc.) of the coronary sinus with respect to the left atrium, the left ventricle, and/or the like. For example, a heart stimulation apparatus 100 having a longer body such as the body 110 may be selected in case the distance between potential pacing locations at the left atrium and at the left ventricle is longer than average while a heart stimulation apparatus 100 having a shorter body 110 may be selected in case the distance between potential pacing locations at the left atrium and at the left ventricle is shorter than average. In another example, an outer diameter of the heart stimulation apparatus 100 (in expanded state) may be selected according to a diameter of the coronary sinus of the subject.

As shown at 504, an intravenous delivery system may be operated to deliver the heart stimulation apparatus 100 to the coronary sinus 106.

The intravenous delivery system may comprise one or more mechanical coupling elements through which the heart stimulation apparatus 100 may be mechanically coupled, attached and/or connected to the intravenous delivery system.

Optionally, the intravenous delivery system may comprise an over tube shaped to accommodate the apparatus 100, specifically the body 110 at least while moved intravenously to the coronary sinus 106.

Reference is now made to FIG. 5B and FIG. 5C, which are schematic illustrations of an exemplary intravenous delivery system adapted for delivering and deploying a heart stimulation apparatus in the coronary sinus of a subject, according to some embodiments of the present invention.

As seen in FIG. 5B, an exemplary intravenous delivery system 550 may be used to deliver a heart stimulation apparatus such as the heart stimulation apparatus 100 to its designated implantation location in the coronary sinus 106 and deploying it in the designated location.

The intravenous delivery system 550 may comprise a deflectable catheter 552 designed and adapted to guide the heart stimulation apparatus 100 through the venous system and veins of the subject, typically entering through the femoral artery, and a handle 554 for guiding the catheter 552 through the artery system. The handle 554 may comprise an adjustment dial to control deployment and retraction of the heart stimulation apparatus 100.

The intravenous delivery system 550 may be designed and/or adapted according to what is known in the art. For example, the intravenous delivery system 550 may be designed as an over the wire system in which the catheter 552 may have a size of 14 Fr, 16 Fr, and/or the like to support a 0.035″ guidewire for example. An overall effective length of the intravenous delivery system 550, specifically of the catheter 552 may be in a range of 100-140 cm, for example, 110 cm.

Optionally, electrical wires may connect the intravenous delivery system 550 to the heart stimulation apparatus 100, for example, 38 AWG enameled wires, and/or the like may be routed through the catheter 552 and exit from the body of the subject through an insertion port of the intravenous delivery system 550, for example, at a proximal end of the handle 554, to support external sensing and/or pacing (stimulation). The electrical wires may be further used, during placement and implant of the heart stimulation apparatus 100, to test its functionality, ensure it works properly and/or for programming it, i.e., updating its software and/or firmware. These electrical wires may be pulled back, disconnected from a connector at the apparatus 100, torn, and/or the like when the intravenous delivery system 550 is retracted and pulled out of the subject's body.

For example, a stent-lime heart stimulation apparatus such as the heart stimulation apparatus 100A, in its folded state 100A (1) (compressed, retracted), may be placed on a distal end of the catheter 552 optionally secured in place using one or more mechanical provisions, for example, a protrusion, a cavity, and/or the like which may mechanically couple the retracted heart stimulation apparatus 100A to the catheter 552.

As seen in FIG. 5C, after guided and delivered the to its designated location in the coronary sinus 106, the heart stimulation apparatus 100A may expand to its expanded state 100A (2), for example, self-expand, and implanted in place, for example, anchored to one or more inner walls of the coronary sinus 106.

As described herein before, the heart stimulation apparatus 100A may be comprise a plurality of segments, for example, two which may be connected to each other via one or more mechanical and/or electrical coupling elements, for example, a coiled wore 506 such as the coiled wire 206.

Optionally, each of the segments of the heart stimulation apparatus 100A may be implanted separately and connected to each other in-body during the implantation.

Moreover, the intravenous delivery system may optionally comprise one or more guiding elements configured for mechanical coupling to the body 110 of the heart stimulation apparatus 100. The guiding means may connect to a deployment end of the intravenous delivery system, for example, a catheter, a guide wire, and/or the like and may be fitted with the heart stimulation apparatus 100 which is to be located in the coronary sinus 106.

The guiding means may comprise one or more guiding elements shaped to geometrically rotationally interlock with the body 110 and/or body 110 is designed for such interlocking. The guiding element(s) may be operated to rotate and since it is interlocked with the boy 110, the body 110 and optionally the entire apparatus 100 may also rotate accordingly (e.g., where one segment is anchored, such rotation may only affect the unanchored segment). The guiding element may be operated to rotate the body 110 and orient it in the determined angular position.

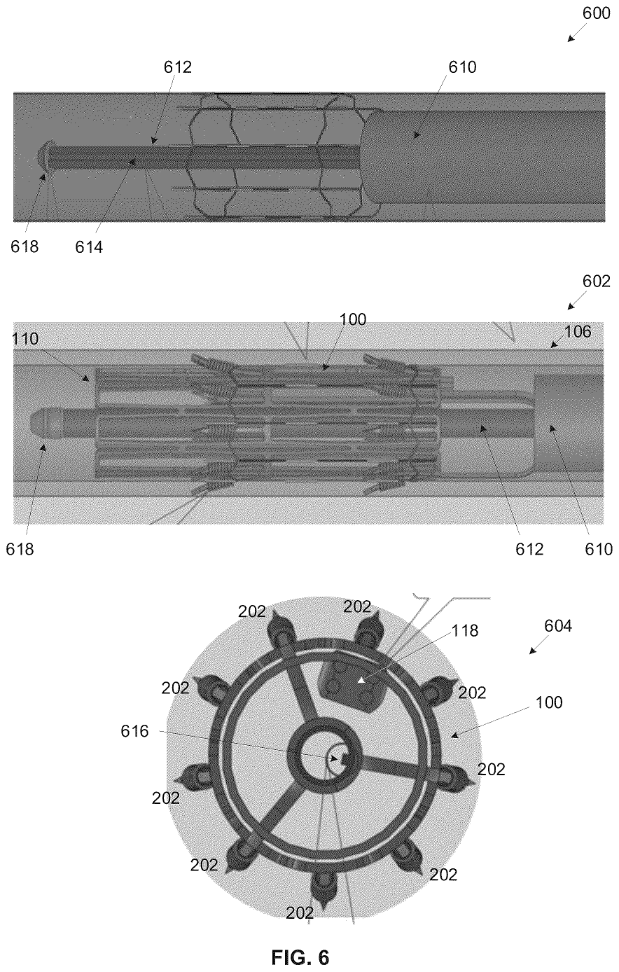

Reference is now made to FIG. 6, which presents schematic illustrations an exemplary guiding element configured and operable for placing an exemplary heart stimulation apparatus in the coronary sinus of a subject, according to some embodiments of the present invention.

As seen in 600, an exemplary intravenous delivery system 610 may be used for delivering and locating an apparatus such as the heart stimulation apparatus 100 in the coronary sinus 106 of a subject for artificially stimulating the heart of the subject. While the drawings may show only small sections of the intravenous delivery system 610, mainly an end section of a catheter of the intravenous delivery system 610, this section is designated intravenous delivery system 610 herein after.

The intravenous delivery system 610 may comprise one or more guiding elements 612 shaped to mechanically connect and interlock with a body such as the body 110 of the heart stimulation apparatus 100. In particular the guiding elements 612 may interlock with the body 110 in the rotational axis around the longitudinal axis of a body such as the body 110 of the heart stimulation apparatus 100 such that when the guiding element 612 is rotated, the body 110 and hence the entire apparatus 100 may also rotate around the longitudinal axis of the body 110.

The guiding element 612 may comprises mechanical provisions shaped to receive and accommodate mating mechanical provisions disposed on the body 110 and interlock with the body 110 in the rotational axis. One or more methods, techniques, and/or designs may be applied for shaping the mechanical provisions of the guiding element 612 and the body 110 to support their interlock with each other.

For example, the mechanical provisions of the guiding element 612 may comprise one or more depressions and the mating mechanical provisions of the body 110 may comprise one or more corresponding protrusions shaped to fit into the depressions of the guiding element 612 and/or vice versa, i.e., one or more depressions may be disposed on the body 110 and one or more corresponding protrusions may be disposed on the guiding element 612. For example, one or more grooves 614 may be created in the guiding element 612 along its longitudinal axis. In a particular example, guiding element 612 includes a single groove along which body 110 can slide.

As seen in 602 and 604, the body 110 of the apparatus, for example, a stent-like body such as the body 110A of an apparatus such as the apparatus 100A may be configured, constructed, and/or adapted to comprise one or more mating mechanical provisions 616, for example, a key 616 (e.g. protrusion, extension, etc.) shaped to fit into the groove(s) 614. When the apparatus 100, for example, an apparatus such as the apparatus 100A having a stent-like body 110A is fitted on the intravenous delivery system 610 for delivery to the coronary sinus 106 of the key 616 may fit into the groove 614 and the body 110A may interlock with the guiding element 612 in the rotational axis.

Moreover, since while interlocked in the rotational axis, the body 110, for example, the body 110A may move (slide) back and forth along the longitudinal axis of the guiding element 612, the guiding element 612 may optionally or alternatively comprise a limiting element 618 disposed at a distal end of the guiding element 612 for limiting a movement of the body 110 along the longitudinal groove 614. The limiting element 618 may be shaped, designed, and/or disposed according to one or more designs, constructions, and/or shapes, for example, a cap and/or a nosecone.

The limiting element 618 may be removed, folded, flattened and/or retracted into the intravenous delivery system 610 in order to release the body 110 from the intravenous delivery system 610 when the heart stimulation apparatus 100 is deployed in a selected location in the coronary sinus.

FIG. 6 also shows an example where a heart stimulation apparatus 100 with electrodes 202 in all direction are provided, but a heart stimulation apparatus 100 with circumferential sectors of electrodes may be also used, for example.

Reference is made once again to FIG. 5A.

As shown at 506, which is an optional step, mapping may be done to map an effect and/or response of the muscle tissue of the left atrium 102 and/or the left ventricle 104 to electrical stimulation in order to identify locations in the left atrium 102 and/or the left ventricle 104 where pacing and/or sensing may be useful. The effect and/or response of the muscle tissue may include, for example, electrical signals which may be captured, measurement and analyzed. In another example, the effect and/or response of the muscle tissue may include, contraction of the muscle which may be monitored, measured and evaluated.

Based on the mapping, an electrical activity map may be created for the tissue near the coronary sinus 106, in particular the tissue of the left atrium 102 and/or the left ventricle 104.

A part from mapping locations in the left atrium 102 and/or the left ventricle 104 where pacing and/or sensing may be useful, the mapping may be further applied to drive pacing electrical signals having various signal parameters (e.g., amplitude, timing, etc.) and evaluating the effect and/or response of the muscle tissue of the left atrium 102 and/or the left ventricle 104 to the varying pacing electrical signals.

While the mapping is conducted to identify locations in the left atrium 102 and/or the left ventricle 104 where pacing and/or sensing may be useful, the mapping may also indicate locations in which electrification and/or stimulation should be avoided. For example, there may be locations in which driving pacing electrical signals may capture the phrenic nerve. In another may be locations in which driving pacing electrical signals may stimulate both the left atrium 102 and the left ventricle 104 simultaneously. The mapping of the effect and/or response of the muscle tissue of the left atrium 102 and/or the left ventricle 104 may reveal such locations which may be mapped accordingly and avoided.

In order to map the stimulation effect and/or response of the muscle tissue of the left atrium 102 and/or the left ventricle 104, the intravenous delivery system 610 may comprise one or more mapping elements and/or arrays which may be deployed, for example, spread, placed, and/or the like to drive and optionally sense stimulation electrical signals at various locations of the left atrium 102 and/or the left ventricle 104 from within the coronary sinus 106.

The mapping elements may be operated to drive stimulation electrical signals at a plurality of location of the left atrium 102 and/or the left ventricle 104 which may be evaluated and/or considered as candidate target locations for the apparatus 100 to drive and/or sense pacing electrical signals. The stimulation electrical signals and/or a response of the muscle tissue of the left atrium 102 and/or the left ventricle 104 to the stimulation electrical signals may be analyzed, for example manually, using an automated mapping tool, computing system, and/or the like ) to identify the locations in the left atrium 102 and/or the left ventricle 104 which may be useful for pacing and/or sensing.

Once the useful pacing and/or sensing locations are identified, an angular position of the heart stimulation apparatus 100 may be selected accordingly to position the atrial and ventricular struts to penetrate these locations.

Reference is now made to FIG. 7A and FIG. 7B, which are schematic illustrations of exemplary mapping elements of an intravenous delivery system configured and operable for placing an exemplary heart stimulation apparatus in the coronary sinus of a subject, according to some embodiments of the present invention.

Illustrations 700, 702 and 704 show several exemplary mapping elements 710 of an intravenous delivery system such as the intravenous delivery system 610 configured and operated to deliver a heart stimulation apparatus such as the apparatus 100 to the coronary sinus 106 and locate (place) the apparatus 100 in a determined location at a determined angular position around its longitudinal axis.

Each mapping element 710 may comprise a plurality of electrodes 712 which may be used to drive and/or sense electrical signals for stimulating the muscle tissue of the left atrium 102 and/or the left ventricle 104.

As seen in illustration 700, a first exemplary mapping element 710A may consist of a scaffold structure in which a plurality electrodes 712A may be deployed. Each of the electrodes 712A may be configured and/or operated to drive and/or sense stimulation electrical signals. As seen in illustration 702, another exemplary mapping element 710B may consist a coil structure in which a plurality of electrode elements 712B are deployed where each electrode 712B may be configured and/or operated to drive and/or sense stimulation electrical signals. As seen in illustration 705, 706, and 708 another exemplary mapping element 710C may consist a fan-like structure having a plurality of “arms” deployed with a plurality of electrodes 712C each configured and/or operated to drive and/or sense stimulation electrical signals.

For mapping purposes, it may be sufficient to place the mapping elements 710 (e.g. 710A, 710B, 710C) in the coronary sinus 106 and use the sensed signals at each electrode 712 to choose desired pacing and/or sensing locations in the coronary sinus 106. A suitably sized and/or shaped heart stimulation apparatus 100 may be selected for implantation according to the mapping.

Optionally, the mapping system (element) may be repositioned, for example, if it is too short to cover the entire coronary sinus where mapping is needed or even if it is merely too short to bridge the desired atrial and ventricular pacing regions. Mapping may be repeated one or more times. In one embodiment, the mapping system (e.g., one or more electrodes) is pulled and/or rotated while continuously measuring. The location of the mapping array may be determined based on the movement and/or rotation of a locked guide element and/or using one or more position sensors thereon.

A suitable apparatus 100 which matches the desired and/or useful pacing and/or sensing locations is selected.

Reference is made once again to FIG. 5A.

As shown at 508, which is an optional step complementing optional step 506, in case a mapping element(s) was deployed, the intravenous delivery system 610 may be operated to retract, close, fold, flatten, and/or collect the mapping element(s) so that it will not interfere with positioning the body 110 in a selected location in the coronary sinus 106.

Alternatively, the mapping array 710 may be remained (left) in the body of the subject after the heart stimulation apparatus 100 is deployed.

As shown at 510, the intravenous delivery system 610 may be operated to rotate the guiding element 612, and the interlocked body 110 of the apparatus 100, according to the selected angular position determined based on the mapping of the useful pacing and/or sensing locations at the left atrium 102 and the left ventricle 104. This means that when the apparatus 100 delivered by the intravenous delivery system 610 arrives at its determined location in the coronary sinus, its orientation can match the one used by mapping array 613(e.g., due to the guiding element 612).

As shown at 512, once oriented according to the selected angular position, the intravenous delivery system 610 may be operated to position (place) the apparatus 100, specifically the body 110 in the coronary sinus 106 in the selected angular and/or axial position.

For example, in case of an apparatus such as the apparatus 100A comprising a stent-like body such as the body 110A, the intravenous delivery system 610 may be operated to expand the stent-like body 110A to its expanded state such that the body 110A may contact at least partially the walls of the coronary sinus 106. For example, the stent-like body 110A may be expanded by retracting a protective over-tube confining the stent-like body 110A in its folded (compressed, retracted) state. In another example, the apparatus 100 may be delivered using an expanding balloon.

In the expanded state of the body 110A, one or more atrial struts such as the atrial struts 202 may penetrate through the wall of the coronary sinus 106 into the left atrium 102 and one or more ventricular struts such as the ventricular struts 204 may penetrate through the wall of the coronary sinus 106 into the left ventricle 104. As such, at least some of the atrial struts 202 and the ventricular struts 204 may be embedded and buried at least partially in the muscle tissue of the left atrium 102 and the left ventricular ventricle 104 respectively.

Moreover, the body 110 of the apparatus 100 may be anchored to robustly secure it in its determined location in the coronary sinus 106. While the anchoring may be done through one or more of the atrial struts 202 and/or one or more of the ventricular struts 204, the body 110 of the heart stimulation apparatus 100 may be anchored through the use of one or more anchoring elements of the heart stimulation apparatus 100if available.

Optionally, for apparatuses 100 constructed of multiple connected segments, for example, a stent-like heart stimulation apparatus such as the apparatus 100A, one or more of the segments may be each delivered, deployed, and/or implanted separately and connected to the other segments in-body, i.e., inside the body of the subject, during, and/or after implanted in their designated location in the coronary sinus 106.

For example, the segments of the apparatuses 100 may be connected via one or more flexible mechanical coupling elements, for example, a wire, a coil such as the coil 206, and/or the like. In such case, a first segment of the apparatus 100, for example, a segment such as the segment 110A 1 may be positioned, placed, and implanted first. Due to flexibility of the mechanical coupling elements, a second segment of the apparatus 100, for example, a segment such as the segment 110A2 may be then positioned, placed, and implanted significantly freely and uninterrupted by the implanted first segment.

The segmented structure of the apparatus 100 and the flexible mechanical connection between the segments may significantly increase maneuverability and flexibility during delivery of the apparatus 100 through the subject's blood vessels, for example, improve passage through bends, and/or the like.

As shown at 514, after placed in its selected location in the coronary sinus 106, the heart stimulation apparatus 100 may be optionally calibrated and/or tested.

Calibration and/or testing of the heart stimulation apparatus 100 may comprise for example, configuring, calibrating and/or testing the controller assembly 116 and/or one or more of its components, for example, the pacing control logic, one or more of the sensors 130, the communication module 132, and/or the like.

Calibration and/or testing of the heart stimulation apparatus 100 may be done using one or more programming, calibration, and/or testing devices (designated programmer herein after) communicating with the controller assembly 116 after the heart stimulation apparatus 100 is implanted in the coronary sinus 106. Optionally, the programmer may be provided in a kit used to calibrate and/or test the heart stimulation apparatus 100. Moreover, the kit may further include the heart stimulation apparatus 100 itself such that during and/or after implantation of the heart stimulation apparatus 100, the programmer may be used to configure, calibrate and/or test the heart stimulation apparatus 100.

The programmer may communicate with the controller assembly 116 of the implanted apparatus 100, for example, via the wireless communication module 132. In another example, the programmer may communicate with the controller assembly 116 via the intravenous delivery system 610 used to deliver the apparatus to its designated location in the coronary sinus 106 before the intravenous delivery system 610 is removed from the subject's body. In such case the programmer using the intravenous delivery system 610 and/or integrated with it may communicate with the controller assembly 116 via one or more communication channels available at the apparatus 100, for example, at the communication module 132. In another example, the intravenous delivery system 610 may be inserted and/or operated in a subsequent procedure after the apparatus 100 is already implanted in the coronary sinus 106.

Calibration and testing of the pacing control logic may include, for example, setting one or more signal parameters of the pacing signals driven to the left atrium 102 and/or the left ventricle 104, for example, a timing (e.g., frequency, cycle, width, etc.), an amplitude, a pulse shape and/or the like.

During calibration, electrical sensing from the electrode assemblies 112 and/or 114 may be used to identify which atrial and/or ventricular struts are best for driving the pacing electrical signals.

Optionally, calibration of the heart stimulation apparatus 100 may be done according the mapping of the effect and/or response of the muscle tissue of the left atrium 102 and/or the left ventricle 104, for example, based on the electrical activity map generated during mapping step 506.

For example, assuming that multiple atrial struts 202 penetrate the muscle tissue of the left atrium 102. Further assuming that, based on the mapping, stimulating the left atrium 102 via one or more some specific atrial struts 202 have increased effect compared to the other atrial struts 202. In such case the controller assembly 116, specifically the pacing control logic may be calibrated to drive pacing electrical signals only to the increased effect atrial struts 202. The same calibration may be applied for the ventricular struts 204 with respect to the left ventricle 104.

In another example, assuming that during the mapping, it was detected that a certain, relatively low, signal amplitude is sufficient for effective pacing of the left atrium 102. In such case, the controller assembly 116, may be calibrated to drive pacing electrical signals having the certain signal amplitude to the first electrode assembly 112 for stimulating the left atrium 102. A gain, the same calibration may be applied for the second electrode assembly 114 for stimulating the left ventricle 104. In another example, assuming that during the mapping, it was determined that a certain pulse timing of pacing electrical signals is most effective for pacing the subject's heart. In such case, the controller assembly 116, may be calibrated to drive pacing signals having the certain pulse timing to the first electrode assembly 112 and/or to the second electrode assembly 114 for stimulating the left atrium 102 and the left ventricle 104 respectively.

The pacing operation of the controller assembly 116 and optionally one or more other functions of the controller assembly 116, for example, pacing signals sensing, motion sensing, communication, and/or the like may be tested to verify their proper operation.

As shown at 516, the heart stimulation apparatus 100 may be further configured to wirelessly communicate, via the communication model 132 to synchronize with one or more other pacing devices deployed in the subject for pacing and/or sensing pacing signals of the subject's heart. These other pacing device(s) may of course be capable to communicate wirelessly and establish a communication session with the apparatus 100 to exchange sensing and/or stimulation (pacing) data which may be used by the distinct pacing devices to synchronize with each other accordingly.

Such synchronization between the heart stimulation apparatus 100 and the other pacing device(s) may enable coherent and orderly stimulation of the subject's heart which may be highly advantageous in pone or more aspects, for example, increase pacing effectivity, reduce potential pacing irregularities, reduce damage to the heart's muscle, reduce and potentially eliminate progressive disease and more.

For example, assuming a single chamber pacemaker is deployed to pace the right ventricle of the subject's heart. In such case, the heart stimulation apparatus 100 may be configured to synchronize its pacing electrical signals with those of the single chamber pacemaker thus combined pacing the left atrium 102, the left ventricle and the right ventricle.

Moreover, the heart stimulation apparatus 100 may be configured to operate in conjunction with another pacing device which is partially malfunctioning. For example, a single chamber pacemaker, a dual chamber pacemaker, and/or a CRT deployed to stimulate the subject's heart may be unable to drive pacing signals but may be capable of sensing and capturing sensing information, for example, sensed pacing electrical signals, natural or not. In such cases, the heart stimulation apparatus 100 may be configured to receive sensing information form the other pacing device and adjust, and/or synchronize its pacing electrical signals according to the received sensing information.

Such deployment may enable the heart stimulation apparatus 100 to take advantage of the partially functioning pacing device thus ensuring effective pacing of the subject's heart without the need to remove the partially functioning pacing device and avoiding surgery.

According to some embodiments of the present invention, there is provided a kit comprising the heart stimulation apparatus 100 and an intravenous delivery system adapted and configured to deliver the apparatus 100 to its designated location in the coronary sinus 106 of the subject, position it, and deploy and/or implant the apparatus 100 in its designated location, and/or orientation.

This kit may be used, for example, by interventional cardiologists seeking less invasive option compared to current trans venous pacemakers which may requires high surgical skills to create skin incision and preparation of sub cutaneous pocket, and the use of trans venous leads which may cause potential harms during and/or after the procedure. In another example, the kit may be used by electrophysiologists for further development of device implants and other cardiac procedures requiring rhythm management. In another example, the kit may be used by inventors developing heart sensing and/or stimulation (pacing) technology for example, for proof of concept, feasibility and performance validation, and/or the like.

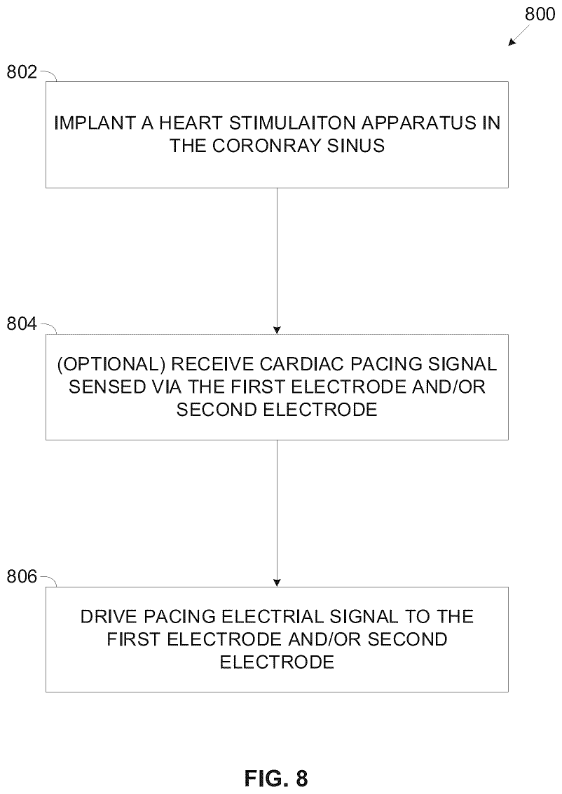

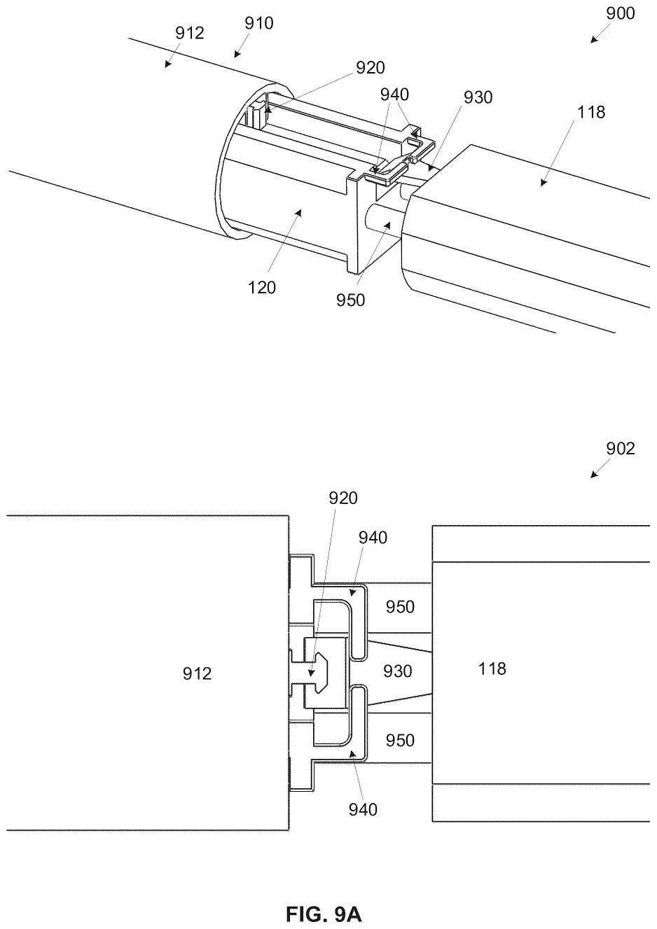

Reference is now made to FIG. 8, which is a flowchart of an exemplary process for artificially stimulating the left atrium and/or the left ventricle of a subject using a heart stimulation apparatus deployed in the coronary sinus of the subject, according to some embodiments of the present invention.

An exemplary process 800 may be executed to pace (or otherwise stimulate) the heart of a subject by artificially stimulating the left atrium and/or the left ventricle of the subset using a heart stimulation apparatus such as the apparatus 100 located (deployed) in the coronary sinus 106 of the subject.

As described herein before, the apparatus 100 may comprise a body such as the body 110 to which two electrode assemblies are mechanically coupled. A first electrode assembly such as the first electrode assembly 112 for electrifying the left atrium 102 and a second electrode assembly such as the second electrode assembly 114 for electrifying the left atrium 102. The first electrode assembly 112 may comprise a plurality of atrial struts such as the atrial struts 202 oriented to penetrate the left atrium 102 when the apparatus 100 is located in the coronary sinus 106 and the second electrode assembly 114 may comprise a plurality of ventricular struts such as the ventricular struts 204 oriented to penetrate the left ventricle 104 when the apparatus 100 is located in the coronary sinus 106.

As shown at 802, the apparatus 100 is implanted and placed in the coronary sinus 106 of the subject as described in the process 500. Moreover, the apparatus 100 may be optionally calibrated, tested and/or synchronized to one or more other pacing devices as also described in the process 500, in steps 514 and 516.

After deployed and anchored in its determined location and orientation in the coronary sinus 106, the apparatus 100 having at least some of its atrial struts 202 and ventricular struts 204 embedded and buried at least partially in the muscle tissue of the left atrium 102 and the left ventricle 104 respectively, may be used.

Moreover, as described herein before, as described herein before, after deployed the apparatus 100 may be calibrated and optionally synchronized with one or more other pacing devices deployed to pace and/or sense the heart of the subject.

As shown at 804, which is an optional step, a controller assembly such as the controller assembly 116 of the apparatus 100 may optionally receive cardiac signals sensed via one or more of the atrial struts 202 and/or one or more of the ventricular struts 204 and/or using other electrodes on body 110. For example, the controller assembly 116 may receive one or cardiac pacing signals naturally generated by the left atrium 102 which are sensed by one or more of the atrial struts 202. In another example, the controller assembly 116 may receive one or cardiac pacing signals naturally generated by the left ventricle 104 which are sensed by one or more of the ventricular struts 204.

As shown at 806, the controller assembly 116 may drive one or more pacing electrical signals to the first electrode assembly 112 for electrifying the left atrium 102 and/or to the second electrode assembly 114 for electrifying the left ventricle 104.

The controller assembly 116 may apply one or more schemes, methods, and/or algorithms as known in the art for driving the pacing electrical signals.

For example, every predefined time period, the controller assembly 116 may drive a first pacing electrical signal to the first electrode assembly 112 followed by a second pacing electrical signal driven to the second electrode assembly 114.

In another example, the controller assembly 116 may drive a first pacing electrical signal to the first electrode assembly 112 to stimulate the left atrium 102 in case no natural cardiac pacing signal is sensed and detected in the left atrium 102 during a predefined time interval. However, in case a natural cardiac pacing signal is sensed and detected in the left atrium 102, the controller assembly 116 may inhibit pacing electrical signals to the first electrode assembly for a predefined time interval, for example, until a subsequent cycle in which the natural cardiac pacing signal is checked again. The same scheme may be applied for driving pacing electrical signals to the second electrode assembly 114 for stimulating the left ventricle 104.

According to some embodiments of the present invention, one or more batteries 120 of the apparatus 100 may be replaced while the apparatus 100 is located and anchored in its determined location in the coronary sinus 106 of the subject.

A battery retrieval device for intravenously retrieving the battery(s) 120 of the heart stimulation apparatus 100 may be typically attached, fitted, integrated and/or otherwise mechanically and functionally coupled to one or more intravenous systems which may be typically used for intravenously delivering devices, medical treatment, surgery and/or other procedures.

The battery retrieval device may be configured and operable to arrive intravenously to the apparatus 100 located in a blood vessel of the subject, for example, the coronary sinus 106, release one or more batteries 120 of the apparatus 100 by unlocking a snap-fit element attaching the respective battery 120 to the capsule 118 of the apparatus 100, and retrieve the released battery(s) 120.

The snap-fit element which detachably mechanically couples the battery 120 to the capsule 118 may be implemented using one or more mechanical concepts, methods, configurations and/or implementations and the battery retrieval device may be configured and operated accordingly. While an exemplary snap-fit element is described herein, other snap-fit designs and/or other interference-locking designs may be used as well.

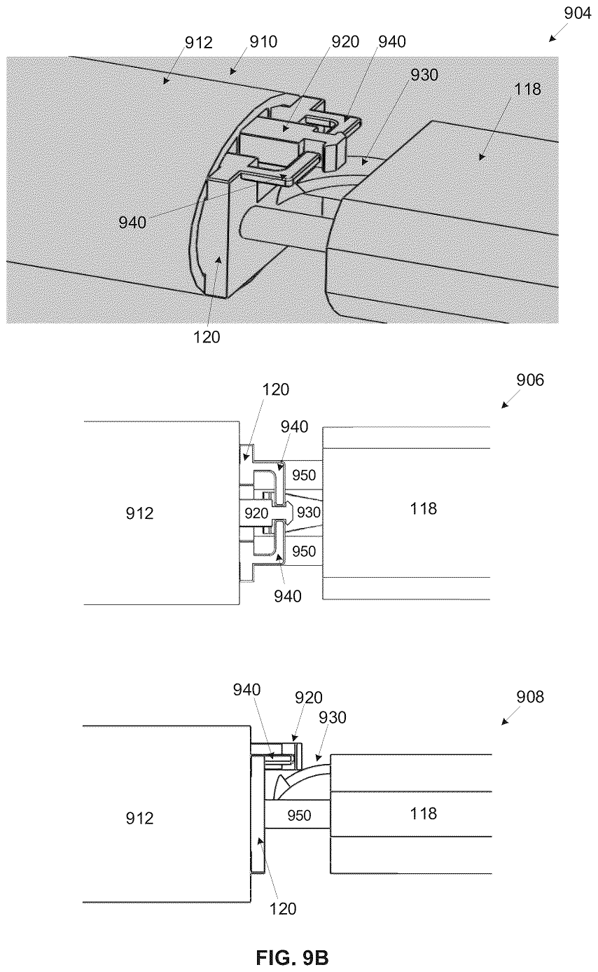

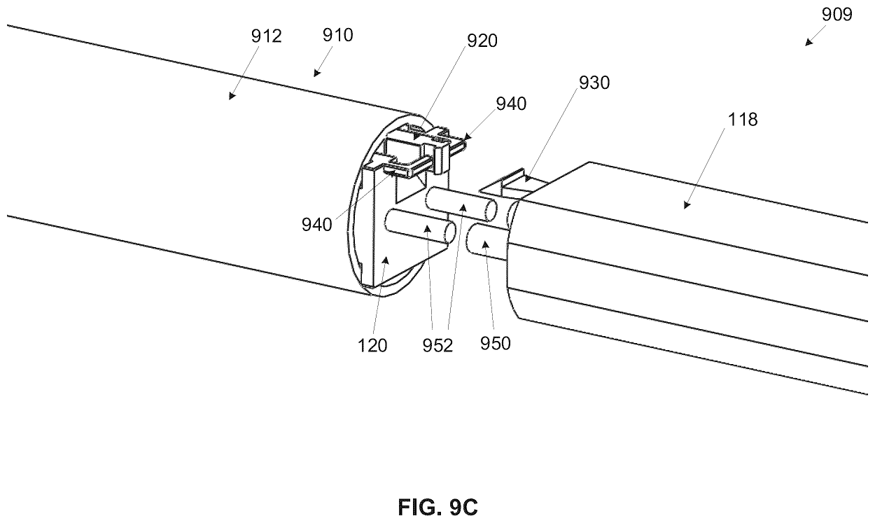

Reference is now made to FIG. 9A, FIG. 9B and FIG. 9C, which are schematic illustrations of an exemplary battery retrieval device configured and operable for retrieving one or more batteries of an exemplary heart stimulation apparatus deployed in the coronary sinus of a subject, according to some embodiments of the present invention.

As seen in 900, a battery such as the battery 120 of a heart stimulation apparatus such as the apparatus 100 may electrically connect to a capsule such as the capsule 118 via one or more terminals 950 to power a controller assembly such as the controller assembly 116 encapsulated in the capsule 118.

The battery 120 may be connected to the capsule 118 via a snap-fit element 930 disposed on the capsule 118 that may lock with one or more mating elements 940 disposed on the battery 120, for example, L shaped elements shaped, adjusted and/or adapted to fit to the snap-fit element 930.

Alternatively, the snap-fit element 930 may be disposed on the battery 120 and one or more mating elements 940 may be disposed on the capsule 118. Moreover, in some implementations, a first snap-fit element 930 may be disposed on capsule 118 for locking with a first mating element(s) 940 disposed on the battery 120 and a second snap-fit element 930 may be disposed on the battery 120 with a second mating element(s) 940 disposed on the capsule 118.

The snap-fit element 930 may be at least partially elastic such that it may be forced to move downwards to a point where the L-shaped mating elements 940 of the battery 120 may be released in a longitudinal axis from the capsule 118, detaching the battery 120 from the capsule 118.

A battery retrieval device 910 may be fitted mechanically and functionally to an intravenous systems capable of arriving intravenously to the apparatus 100 located in a blood vessel of the subject.

The battery retrieval device 910 may comprise a battery retrieval element 920 which may be operable to detach and release the battery 120 from the capsule 118 by releasing the snap-fit element 930.

Optionally, the battery 120 may be shaped to include alignment provisions for accommodating and aligning the battery retrieval element 920 while the battery retrieval element 920 is advanced towards the snap-fit element 930. For example, the body of the battery 120 may compose a longitudinal groove in which the battery retrieval element 920 may fit to be aligned perpendicularly to the snap-fit element 930.

Optionally, the battery 120 may have an elongated body to host a substantially long grove which may enable improved alignment of the battery retrieval element 920 with respect to the snap-fit element 930.

As seen in 902, the battery retrieval device 910 may be operated to advance the battery retrieval element 920 towards the snap-fit element 930. The battery retrieval device 910 may be designed, shaped, and/or constructed to ensure alignment of the battery retrieval element 920 with the snap-fit element 930, for example, in a lateral axis, in a longitudinal axis, to ensure that the battery retrieval element 920 and the snap-fit element 930 are substantially aligned in space for effective engagement of the battery retrieval element 920 with the snap-fit element 930.

As seen in 904, 906 and 908, the battery retrieval element 920 may push down the snap-fit element 930, releasing the L-shaped mating elements 940 of the battery 120 from the snap-fit element 930 and detaching the battery 120 from the capsule 118. Moreover, the battery retrieval element 920 may be shaped to include mechanical provisions for mechanically attaching to one or more of the mating element(s) 940 of the battery 120 while pushing down the snap-fit element 930.

As seen in 909, the battery retrieval device 910 may be operated to move the battery retrieval element 920 backwards, retrieving the battery 120 that is mechanically attached to the battery retrieval element 920 via the mating element(s) 940. As seen, when the battery retrieval element 920 moves backwards one or more battery electrical terminals 952 may disconnect from the respective electrical terminals 950 of the capsule 118.

Optionally, the battery retrieval device 910 may comprise an over tube shaped to accommodate the battery 120, specifically after retrieved from the apparatus 100.

The battery retrieval device 910 may be further operated to replace the retrieve battery 120 with a new battery 120 by repeating the same sequence in a reverse order.

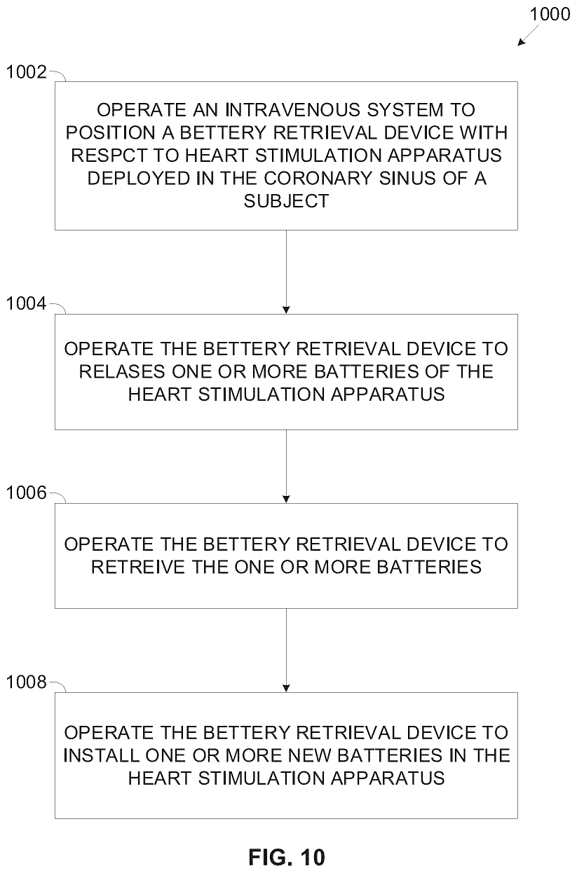

Reference is also made to FIG. 10, which is a flowchart of an exemplary process for replacing one or more batteries of an exemplary heart stimulation apparatus deployed in the coronary sinus of a subject, according to some embodiments of the present invention.

An exemplary process 1000 may be executed to retrieve and optionally replace one or more batteries 120 of the heart stimulation apparatus 100 while the apparatus 100 is located in a blood vessel of the subject, for example, in the coronary sinus 106.

As shown at 1002, the process 800 starts with operating an intravenous system, fitted with the battery retrieval device 910 at its intravenous end, to position the battery retrieval device 910 with respect to the apparatus 100 implanted and located in the a blood vessel of the subject, for example, in the coronary sinus 106.

As shown at 1004, the battery retrieval device 910 may be operated to release one or more of the batteries 120 of the apparatus 100, for example, by advancing a battery retrieval element 920 such as the battery retrieval element 920 towards a snap-fit element such as the snap-fit element 930 attaching the battery(s) 120 to the capsule 118 of the apparatus 100 to unlock the snap-fit element 930.

As shown at 1006, the battery retrieval device 910 may be operated to retrieve the released battery(s) 120. For example, the battery retrieval device 910 may be operated to attach to the released battery(s) and retract it backwards. The battery retrieval device 910 may be further operated to retrieve the released battery(s) and remove it from the body of the subject.

Optionally, the battery retrieval device 910, typically together with the intravenous system, may be operated to attach one or more new batteries to the capsule 118 via the snap-fit element 930 to replace the retrieved battery(s) 120.

It is noted that various embodiments of stimulation apparatus, delivery systems, guiding systems, replacement systems and mapping systems are described, as well as various features therefore. Due to space limit, not every possible combination has been shown. In some cases, a feature is described with respect to one embodiment but not another. It is noted that the application hereby explicitly covers any combination of such apparatus and systems and features.

As described herein before, one or more of the batteries 120 may be rechargeable via a battery charging device which may be very similar to the battery retrieval device 910. The battery charging device may be fitted mechanically and functionally to an intravenous systems capable of arriving intravenously to the apparatus 100 located in the coronary sinus 106 and recharge the battery(s) 120, either wirelessly (e.g., via one or more energy harvesting coils) and/or via one or more contacts and/or terminals disposed on the battery(s) 120 for recharging.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant systems, methods and computer programs will be developed and the scope of the terms stent structure, mapping equipment, and battery technology are intended to include all such new technologies a priori.

As used herein the term “about” refers to ±10 %.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, an instance or an illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An apparatus for artificial stimulation of the heart of a subject, comprising:
an elongated body having a cylindrical structure with flow-lumen therein configured to be located in the coronary sinus of the subject;
a first electrode assembly mechanically coupled to the body and oriented for electrifying the left atrium of the subject when the body is located in the coronary sinus;
a second electrode assembly mechanically coupled to the body sufficiently distant from the first electrode assembly and oriented for electrifying the left ventricle of the subject when the body is located in the coronary sinus and the first electrode assembly is oriented for electrifying the left atrium; and
a controller assembly electrically coupled to the first electrode assembly and to the second electrode assembly, the controller assembly is configured to drive a pacing electrical signal to the first electrode assembly and/or to the second electrode assembly;
wherein the first electrode assembly and/or the second electrode assembly penetrate the left atrium and/or the left ventricle respectively.

2. The apparatus of claim 1, wherein the controller assembly is further configured to:
receive cardiac pacing signal sensed via the first electrode assembly and/or the second electrode assembly, and
drive the pacing electrical signal to the first electrode assembly and/or to the second electrode assembly according to the sensed cardiac pacing data.

3. The apparatus of claim 1, wherein the first electrode assembly comprises a plurality of atrial struts extending from the body for penetrating through a wall of the coronary sinus into muscle tissue of the left atrium and the second electrode assembly comprises a plurality of ventricular struts extending from the body for penetrating through a wall of the coronary sinus into muscle tissue of the left ventricle, each of the plurality of struts comprises at least one electrical lead for delivering the pacing electrical signal.

4. The apparatus of claim 3, wherein the plurality of atrial struts extend from the body in a first radial direction with respect to a longitudinal axis of the body and the plurality of ventricular struts extend from the body in a second radial direction with respect to a longitudinal axis of the body, the first radial direction is offset from the second radial direction to orient the plurality of atrial struts to extend towards the left atrium and orient the plurality of ventricular struts towards the left ventricle when the body is located in the coronary sinus.

5. The apparatus of claim 3, wherein the plurality of atrial struts extend from the body in a plurality of first radial directions covering a predefined first circumferential sector and the plurality of ventricular struts extend from the body in a plurality of second radial directions covering a predefined second circumferential sector, the first circumferential sector is offset from the second circumferential sector to orient the plurality of atrial struts to extend towards the left atrium and orient the plurality of ventricular struts to extend towards the left ventricle when the body is located in the coronary sinus.

6. (canceled)

7. (canceled)

8. The apparatus of claim 3, wherein the at least one electrical lead of at least one of the plurality of atrial struts and/or ventricular struts is extending from an interior bore of the at least one strut.

9. (canceled)

10. (canceled)

11. The apparatus of claim 3, wherein at least some of the plurality of struts are shaped for anchoring the body in place when located in the coronary sinus.

12. The apparatus of claim 1, further comprising at least one anchor element for anchoring the body in place when located in the coronary sinus.

13. The apparatus of claim 1, wherein the body has is a stent-like structure.

14. (canceled)

15. The apparatus of claim 1, wherein the controller assembly is encapsulated in a capsule mechanically coupled to the body, the capsule comprises at least one battery electrically connected to the controller assembly for powering the controller assembly.

16. The apparatus of claim 15, wherein the at least one battery is detachably attached to the capsule via a releasable snap-fit element.

17. (canceled)

18. (canceled)

19. A kit for delivering and positioning an apparatus for artificial stimulation of the heart of a subject, comprising:
an apparatus according to claim 1; and
an intravenous delivery system comprising a guiding element shaped to mechanically interlock with the body and operable to rotate the body for placing the body in a determined angular positon around a longitudinal axis of the body.

20. The kit of claim 19, wherein the guiding element comprises mechanical provisions shaped to receive and accommodate mating mechanical provisions disposed on the body and interlock with the body in a rotational axis around the longitudinal axis of the body to induce rotation of the body when rotating the guiding element.

21. The kit of claim 20, wherein the mechanical provisions of the guiding element comprise at least one depression and the mating mechanical provisions of the body comprise at least one protrusion shaped to fit into the at least one depression and/or vice versa.

22. The kit of claim 19, wherein the guiding element further comprises a limiting element disposed at a distal end of the guiding element for limiting a movement of the body along its longitudinal axis.

23. The kit of claim 19, wherein the intravenous delivery system further comprises at least one mapping element comprising a plurality of mapping electrodes configured to drive stimulating electrical signals to the left atrium and/or to the left ventricle, a muscular activity of the left atrium and/or to the left ventricle in response to the stimulating electrical signals is analyzed to map stimulation effect of the stimulating electrical signals.

24. (canceled)

25. A method of positioning a heart stimulation apparatus in a coronary sinus of a subject, comprising:
mapping a location of the left atrium and the left ventricle of the subject;
operating an intravenous delivery system to deliver a heart stimulation apparatus to the coronary sinus of the subject;
mapping a stimulation effect of muscle tissue of the left atrium and/or the left ventricle based on analysis of a response to stimulation electrical signals injected at a plurality of locations of the left atrium and/or the left ventricle using at least one mapping element coupled to the intravenous delivery system;
selecting an angular position of a guiding element of the intravenous delivery system interlocked with the heart stimulation apparatus according to the mapping;
operating the intravenous delivery system to rotate the guiding element according to the selected angular position to position the heart stimulation apparatus in a determined angular position with respect to the left atrium and the left ventricle; and
operating the intravenous delivery system to place the heart stimulation apparatus in the coronary sinus in the determined angular position such that a first electrode and/or a second electrode of the heart stimulation apparatus penetrate the left atrium and/or the left ventricle respectively.

26. The kit of claim 19, wherein the intravenous delivery system comprises a capsule replacement element operable to replace the at least one capsule.

27. The kit of claim 19, wherein the intravenous delivery system comprises a battery replacement element operable to replace the at least one battery.

28. The method of claim 25, further comprising replacing a capsule of an apparatus for artificial stimulation of the heart of a subject, comprising:
operating an intravenous system to position a capsule replacement device with respect to a heart stimulation apparatus located in the coronary sinus of a subject; and
operating the capsule replacement device to replace at least one capsule of the apparatus.

29. (canceled)
